(19) United States
(12) Patent Application Publication
(10) Pub. No.: US 2025/0161357 A1
(43) Pub. Date: May 22, 2025

(54) HUMAN T CELL RECEPTOR PAIRS REACTIVE WITH HLA-A*02:01 RESTRICTED HUMAN PROSTATIC ACID PHOSPHATASE (PAP) EPITOPES

(71) Applicants: The Regents of the University of California, Oakland, CA (US); Stanford University School of Medicine, Stanford, CA (US); Fred Hutchinson Cancer Center, Seattle, WA (US)

(72) Inventors: Owen N. Witte, Sherman Oaks, CA (US); Zhiyuan Mao, Los Angeles, CA (US); Jami McLaughlin Witte; Pavlo A. Nesterenko, Los Angeles, CA (US); John K. Lee, Seattle, WA (US); Kenan Christopher Garcia, Stanford, CA (US); Xiaojing Chen, Stanford, CA (US); Xiang Zhao, Menlo Park, CA (US)

(73) Assignees: The Regents of the University of California, Oakland, CA (US); Stanford University School of Medicine, Stanford, CA (US); Fred Hutchinson Cancer Center, Seattle, WA (US)

(21) Appl. No.: 18/837,112

(22) PCT Filed: Mar. 2, 2023
(86) PCT No.: PCT/US23/63586

§ 371 Date: Aug. 8, 2024

Related U.S. Application Data
(60) Provisional application No. 63/315,825, filed on Mar. 2, 2022.

(51) Int. Cl. A61K 35/17; A61K 40/11; A61K 40/32; A61K 40/42; C07K 14/725; C12N 5/0783; C12N 15/86; G01N 33/50

(52) U.S. Cl. CPC A61K 35/17; A61K 40/11; A61K 40/32; A61K 40/4252; C07K 14/7051; C12N 5/0636; C12N 15/86; G01N 33/505; A61K 2239/58; C12N 2510/00; C12N 2740/15011; G01N 2800/52

(57) ABSTRACT

Aspects of the present disclosure relate to methods and compositions related to related to the preparation of immune cells, including engineered T cells having T cell receptors that target human prostatic acid phosphatase (PAP) and are useful in prostate cancer therapy.

### CROSS REFERENCE TO RELATED APPLICATIONS

This application claims the benefit under 35 U.S.C. Section 119 (e) of co-pending and commonly-assigned U.S. Provisional Patent Application No. 63/315,825, filed Mar. 2, 2022, entitled “HUMAN T CELL RECEPTOR PAIRS REACTIVE WITH HLA-A*02:01 RESTRICTED HUMAN PROSTATIC ACID PHOSPHATASE (PAP) EPITOPES”, which application is incorporated by reference herein.

### STATEMENT REGARDING FEDERAL FUNDING

This invention was made with government support under Grant Number CA009120, awarded by the National Institutes of Health. The government has certain rights in the invention.

### TECHNICAL FIELD

Embodiments of the disclosure concern at least the fields of immunology, cell biology, molecular biology, and medicine.

### BACKGROUND OF THE INVENTION

Prostate cancer is a disease in which occurs when cells of the prostate gland mutate and begin to multiply out of control. At present, prostate cancer is the second most commonly diagnosed cancer and the fourth leading cause of cancer-related death in men in the developed countries worldwide.

Typical antigens which have been shown to be overexpressed by prostate cancer cells as compared to normal counterparts include proteins such as prostatic acid phosphatase (PAP), prostate-specific antigen (PSA), Prostate-specific membrane antigen (PSMA), and prostate stem cell antigen (PSCA). Prostate cancer cells may spread (metastasize) from the prostate to other parts of the body, especially the bones and lymph nodes. Prostate cancer may cause pain, difficulty in urinating, erectile dysfunction and other symptoms. Typically, prostate cancer develops most frequently in men over fifty, which represent the most common group of patients. However, prostate cancer remains most often undiscovered, even if determination would be possible. Determination of prostate cancer typically occurs by physical examination or by screening blood tests, such as the PSA (prostate specific antigen) test. When suspected to prostate cancer the cancer is typically confirmed by removing a piece of the prostate (biopsy) and examining it under a microscope. Further tests, such as X-rays and bone scans, may be performed to determine whether prostate cancer has spread.

Treatment of prostate cancer still remains an unsolved challenge. Conventional therapy methods may be applied for treatment of prostate cancer such as surgery, radiation therapy, hormonal therapy, occasionally chemotherapy, proton therapy, or some combination of these. However, the age and underlying health of the man as well as the extent of spread, appearance under the microscope, and response of the cancer to initial treatment are important in determining the outcome of the disease. Since prostate cancer is a disease, typically diagnosed in older men, many will die of other causes before a slowly advancing prostate cancer can spread or cause symptoms. This makes treatment selection difficult. The decision whether or not to treat localized prostate cancer (a tumor that is contained within the prostate) with curative intent is a trade-off between the expected beneficial and harmful effects in terms of patient survival and quality of life.

As known in the art, the immune system plays an important role in the treatment and prevention of numerous diseases. According to the present stage of knowledge, various mechanisms are provided by mammalians to protect the organism by identifying and killing, e.g., tumor cells. For the purposes of the present invention, these tumor cells have to be detected and distinguished from the organism's normal (healthy) cells and tissues.

It is an object of the present invention to provide compositions and methods useful for treatment of prostate cancer (PCa).

### SUMMARY OF THE INVENTION

Prostatic acid phosphatase (PAP) is a well-known prostate/prostate cancer antigen and can serve as a target for cancer therapy. As described herein, HLA-A*02:01 restricted PAP epitopes were defined by using multiple physical methods coupled with liquid chromatography mass spectrometry (LC-MS), including mild acid elution, co-immunoprecipitation and secreted-MHC IP based on the ARTEMIS platform. Recovered PAP epitopes were then used to stimulate peripheral mononuclear cells (PBMCs) from over 20 healthy donors. Reactive T cells isolated by recently developed CLInt-seq and TCR alpha/beta sequencing techniques were analyzed by 10× Genomics single cell TCR sequencing. Polynucleotides encoding paired TCR alpha/beta chains were then introduced into normal human T cells and tested for their functionality. Using this methodology, we have discovered 21 TCR alpha/beta polypeptide pairs specifically recognizing and being activated by 7 distinct PAP peptides. All of these 21 TCRs showed reactivity when screened in Jurkat-NFAT-GFP cells with peptide-pulsed K562-A2 cells. At least 7 out of these 21 TCRs can be specifically stained by cognate tetramers. These 7 candidates can be successfully paired and stimulated in human T cells. At least one TCR (PAP-TCR-156) shows significant IFNγ signal and inhibition of target cell growth when coculturing with cells expressing both HLA-A2 and full-length PAP. These discoveries of PAP epitopes and cognate TCR sequences are useful in new cancer immunotherapies and vaccines.

The invention disclosed herein has a number of embodiments that harness the above-noted discoveries. Embodiments of the invention include, for example, compositions of matter comprising a polynucleotide encoding a T cell receptor (TCR) alpha chain polypeptide and/or a TCR beta chain polypeptide; wherein when transduced into and expressed in a CD8″ T cell, the alpha chain polypeptide and/or the TCR beta chain polypeptide can form a T cell receptor that recognizes/binds a polypeptide epitope on human prostatic acid phosphatase (PAP). In typical embodiments of the invention, the polynucleotide is disposed in a vector comprising one or more regulatory sequences or the like for expressing the polypeptide in a cell. Embodiments of the invention further comprise cells transduced with such vectors (e.g., a CD8″ T cell).

In certain embodiments of the invention, the T cell receptor that recognizes/binds PAP targets at least one polypeptide epitope selected from: ILLWQPIPV (SEQ ID NO: 47), TLMSAMTNL (SEQ ID NO: 48), IRSTDVDRTL (SEQ ID NO: 52), IMYSAHDTTV (SEQ ID NO: 55), KVYDPLYCESV (SEQ ID NO: 58), LLLARAASLSL (SEQ ID NO: 59), and LLFFWLDRSVLA (SEQ ID NO: 62). In some embodiments of the invention, the T cell receptor that recognizes/binds PAP recognizes/binds HLA-A*02:01 restricted epitopes. In some embodiments of the invention, a polynucleotide encoding a T cell receptor (TCR) alpha chain polypeptide or a TCR beta chain polypeptide (e.g. a TCR encoded by SEQ ID NO: 1-SEQ ID NO: 42) is engineered to introduce one or more mutations into a TCR polypeptide selected provide the TCR with a higher target killing sensitivity, typically while retaining physiological affinity (e.g. a catch bond mutation). In certain embodiments of the invention, the polynucleotide encoding a T cell receptor (TCR) alpha chain polypeptide or a TCR beta chain polypeptide encodes a segment of at least 5 or at least 10 amino acids of an Alpha V region polypeptide sequence and/or a Beta V region polypeptide sequence shown in Table A and/or Table B.

Embodiments of the invention also include methods of inhibiting growth of a prostate cancer cell. Typically these methods comprise combining the prostate cancer cell with a CD8″ T cell transduced with a polynucleotide encoding a T cell receptor (TCR) alpha chain polypeptide and a TCR beta chain polypeptide; wherein when transduced into and expressed in a CD8″ T cell, the alpha chain polypeptide and the TCR beta chain polypeptide can form a T cell receptor that recognizes/binds a polypeptide epitope on human prostatic acid phosphatase (PAP). In certain embodiments of the invention, the polynucleotide encoding a T cell receptor (TCR) alpha chain polypeptide or a TCR beta chain polypeptide encodes an Alpha CDR3 region polypeptide sequence and/or a Beta V region polypeptide sequence shown in Table A; and/or the polynucleotide is one shown in Table B. Typically in these methods, polynucleotides encoding the TCR alpha/beta polypeptide pairs are transduced into CD8⁺ T cells which then are combined with the prostate cancer cells in vivo so as to treat an individual suffering from prostate cancer.

Embodiments of the invention further include methods of assessing a patient immune response to a prostate cancer or prostate cancer vaccination. Typically these methods comprise observing the induction or activation of T cells obtained from a patient having a prostate cancer or prostate cancer vaccination, wherein the induction or activation of T cells is observed in response to the T cell's exposure to a polypeptide epitope present on human prostatic acid phosphatase (PAP); and an observed induction or activation of T cells provides evidence of patient immune response to prostate cancer or prostate cancer vaccination. Related embodiments of the invention include methods of assessing a patient immune response to a prostate cancer or prostate cancer vaccination. Typically these methods comprise observing the presence of TCR polypeptide sequences disclosed herein or related TCR polypeptide sequences obtained from a patient having a prostate cancer or prostate cancer vaccination, wherein the presence of TCR polypeptide sequences disclosed herein or related TCR polypeptide sequences evidence of patient immune response to a prostate cancer or prostate cancer vaccination. Yet another embodiment of the invention is a method of generating a peptide-MHC multimer composition useful to assess a patient T cell responses to a prostate cancer or prostate cancer vaccination, the method comprising combining or coupling at least one polypeptide epitopes present on human prostatic acid phosphatase (PAP) with an MHC multimer (e.g. an oligomeric forms of an MHC molecule) so that the peptide-MHC multimer composition is made.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1: Diagram of overall project flowchart and summary of TCR screening process. (Left) Both mono allele and multi allele HLA A0201 cell lines were processed by three different physical methods to extract peptides on MHC I. Sequences of these peptides were then identified by LC MS in result of 27 distinct PAP epitopes. (Right) Peptides were then screened on PBMCs from >20 individuals and reactive clones were identified using CLint seq protocol (see, e.g., Utility: PCT Application Serial No. PCT/US20/49055, which is incorporated herein by reference).

FIGS. 2A-2B: Schematic of and data from T2 stabilization assays designed to assess stability of peptide-MHC I. FIG. 2(a) Schematic for the overall process of T2 assays; FIG. 2(b) Graphed data showing the slopes of natural log of A2 fluorescent intensity vs diluted peptide concentration of various PAP peptides. Positive candidates in the T2 assays shown in FIG. 2B are PAP-A2-14, PAP-A2-20, PAP-A2-21, PAP-A2-22, PAP-A2-25, and PAP-A2-27.

FIG. 3: Schematic of and data from studies using A secreted form of MHC I single chain trimer to assess stability of peptide-MHC. FIG. 3(a) Diagram of the SCT constructs; FIG. 3(b) SDS-PAGE gel results of the relative yield of each PAP SCTs comparing to positive control (+) WT1 peptide RMFPNAPYL.

FIG. 4: Schematic of and data from studies testing candidate TCRs in Jurkat-NFAT-GFP for rapid screening. FIG. 4(a) schematic illustration of the Jurkat-NFAT-GFP system for TCR screening. FIG. 4(b) Example (TCR-218) FACS results of Jurkat-NFAT-GFP screening; Top: DMSO as negative control with TCR-218; Bottom: PAP-A2-21 as positive hit with TCR-218.

FIG. 5: Data from functional tests of candidate TCRs observed with various methods. FIG. 5(a) IFNγ results of different TCR constructs on PBMCs with peptide dilution; F5: positive control against MARTI peptide (EAAGIGILTV); NGFR: negative control with DMSO. FIG. 5(b) IFNγ results of candidate TCR constructs on PBMCs with cell lines with or without full-length PAP; black bars: TCR-engineered PBMCs with K562-A2; grey bars: TCR-engineered PBMCs with K562-A2-PAP. FIG. 5(c) Cytotoxicity curve of TCR-156 by incucyte using total GFP signals of target cells to quantify target cell number; blue: K562-A2 target cells with TCR-156 engineered PBMCs; red: K562-A2-PAP target cells with TCR-156 engineered PBMCs.

FIGS. 6A-6D: Data from functional tests of TCR mutants observed with various methods. FIG. 6A provides data from studies of mutants of PAP-TCR-156 (see Table B) showing that the introduction of substitution mutations can enhance the potency of TCRs such as PAP-TCR-156 without losing specificity. FIG. 6B provides data from studies of PAP-TCR-156 mutants killing K62 A2 cells without PAP (left panel) and with PAP (right Panel) showing that the introduction of mutations can enhance cytotoxicity of TCRs such as PAP-TCR-156 without losing specificity. FIG. 6C provides data from studies showing PAP-TCR-156 mutant specific cytotoxicity on prostate cancer cell lines overexpressing PAP and HLA-A2 (left panel PC3 control cells, right panel PC3 cells expressing PAP). FIG. 6D provides data from studies of PAP-TCR-156 mutants showing that these embodiments of the invention exhibit cytotoxicity on PC3-A2-PAP at lower E: T ratios (4:1 ratio left panel, 1:1 ratio right panel).

FIG. 7: Data from functional screening assays of TCR156 variants showing enhanced functions in PAP peptide titration assays. The left panel shows data from unmutated TCR156 (wt) and TCR156 variants 156-29, 156-30, 156-31, 156-32, 156-33, and 156-34; and the right panel shows data from unmutated TCR156 (wt) and TCR156 variants 156-35, 156-36, 156-37, 156-38, and 156-39.

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention. Certain aspects of the invention disclosed below are also found in Mao et al., Proc Natl Acad Sci USA. 2022 Aug. 2; 119 (31) (hereinafter “Mao et al.”), the contents of which are incorporated by reference.

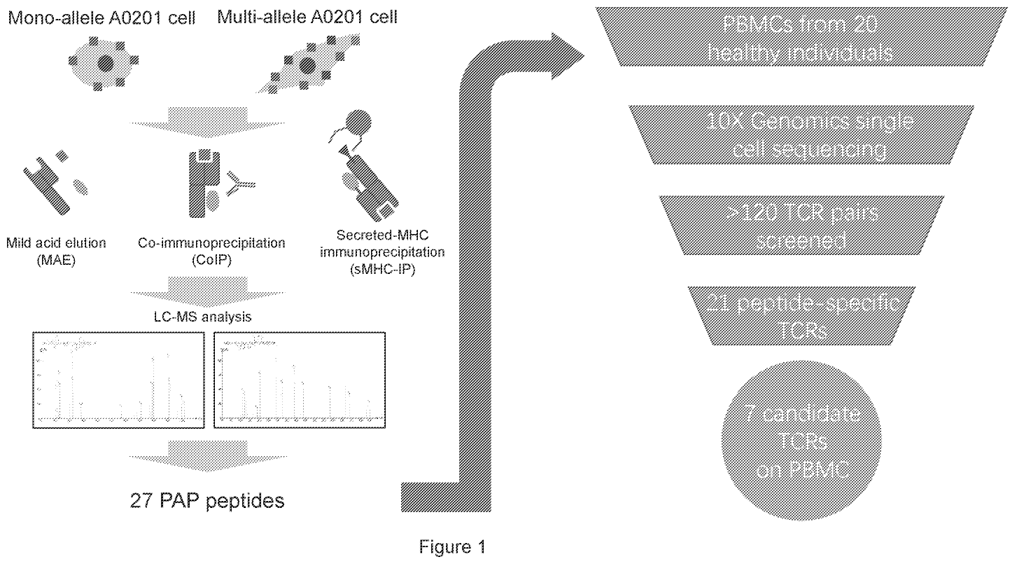

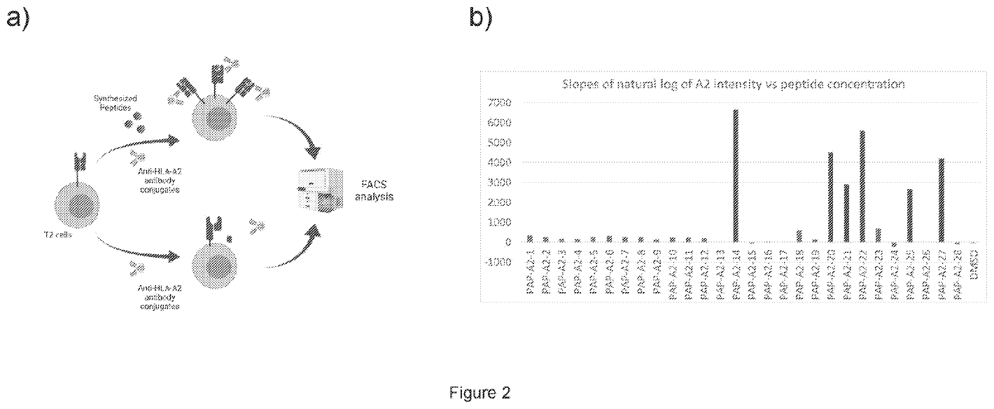

Prostatic acid phosphatase (PAP) is a well-known prostate/prostate cancer antigen and can serve as a target for cancer therapy (Kantoff et al, NEJM, 2010 Jul. 29; 363 (5): 411-22). HLA-A*02:01 restricted PAP epitopes were defined by using multiple physical methods coupled with liquid chromatography mass spectrometry (LC-MS), including mild acid elution (MAE), co-immunoprecipitation (CoIP) and secreted-MHC IP (sMHC-IP) based on the ARTEMIS platform (FIG. 1). 27 candidate PAP peptides were identified in total from all three methods (Table 1 in Mao et al.). Recovered PAP epitopes were then used to stimulate peripheral mononuclear cells (PBMCs) from over 20 healthy donors. Reactive T cells isolated by recently developed CLInt-seq and TCR alpha/beta sequencing techniques were analyzed by 10× Genomics single cell TCR sequencing (FIG. 1) (Nesterenko et al, PNAS Mar. 30, 2021 118 (13) e2100106118). Paired TCR alpha/beta chains were then introduced into normal human T cells and tested for their functionality. We have recovered 21 TCR alpha/beta pairs specifically recognizing and being activated in Jurkat-NFAT-GFP system by 7 distinct PAP peptides from our previous findings (FIG. 2; Table 2,3 in Mao et al.). At least 7 out of these 21 TCRs can be specifically stained by cognate tetramers. These 7 candidates can be successfully paired and stimulated in human T cells. This knowledge of PAP epitopes and cognate TCR sequences can potentially be used to develop new cancer immunotherapy and vaccines.

Embodiments of the invention include compositions of matter comprising one or more vectors comprising the TCR polynucleotides disclosed herein. A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Typically, the vector is an expression vector. The term “expression” as used herein is defined as the transcription and/or translation of a particular nucleotide sequence driven by its promoter. In this context, the term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

Typically, a composition of the invention comprises one or more Vα/Vβ polynucleotides, for example a polynucleotide encoding a TCR Vα polypeptide in combination with a polynucleotide encoding a TCR VB polypeptide such that a Vα/Vβ TCR can be expressed on the surface of a mammalian cell (e.g., a CD8+ T cell) transduced with the vector(s), wherein the Vα/Vβ TCR recognizes a PAP peptide associated with a HLA. The term “transduced” or “transfected” or “transformed” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

In another aspect, the invention includes a method for generating a modified T cell comprising introducing one or more nucleic acids (e.g., nucleic acids disposed within a lentiviral vector) encoding a TCR disclosed herein into a T cell (e.g. a CD8⁺ T cell obtained from an individual diagnosed with a cancer that expresses a PAP epitope recognized by a TCR). The present invention also includes modified T cells with downregulated or knocked out gene expression (e.g., a modified T cell having a knocked out endogenous T cell receptor and an exogenous/introduced T cell receptor that recognizes a PAP peptide associated with a HLA). The term “knockdown” as used herein refers to a decrease in gene expression of one or more genes. The term “knockout” as used herein refers to the ablation of gene expression of one or more genes.

The modified T cells described herein may be included in a composition for use in a therapeutic regimen. The composition may include a pharmaceutical composition and further include a pharmaceutically acceptable carrier. A therapeutically effective amount of the pharmaceutical composition comprising the modified T cells may be administered. Pharmaceutical compositions of the present invention may comprise the modified T cell as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Adoptive immunotherapy with T cells harboring antigen-specific TCRs have therapeutic potential in the treatment of cancers. Gene-engineering of CD 8⁺ T cells with a specific TCR has the advantage of redirecting the T cell to a selected antigen such as an PAP epitope recognized by a TCR. In this context, in one aspect, the invention includes methods for stimulating a T cell-mediated immune response to a target cell or tissue in a subject comprising administering to a subject an effective amount of a modified CD 8⁺ T cell. In this embodiment, the CD8⁺ T cell is modified as described elsewhere herein. Embodiments of the invention also include administering multiple modified CD 8⁺ T cells that target multiple PAP epitopes. For example, embodiments of the invention include administering at least two different modified CD8⁺ T cells, for example a first modified CD8⁺ T cell that targets a PAP peptide associated with a first human leukocyte antigen human leukocyte antigen in combination with a second CD8⁺ T cells that targets a PAP peptide associated with second human leukocyte antigen.

Embodiments of the invention include compositions of matter comprising a polynucleotide encoding a T cell receptor (TCR) alpha chain polypeptide and/or a TCR beta chain polypeptide; wherein the polynucleotide is disposed in a vector, and when the vector is transduced into a CD8⁺ T cell, the alpha chain polypeptide and/or the TCR beta chain polypeptide encoded by the polynucleotide form a T cell receptor that recognizes a polypeptide epitope of human prostatic acid phosphatase (PAP). In certain embodiments of the invention, the T cell receptor recognizes a polypeptide epitope of human prostatic acid phosphatase in combination with human leukocyte antigen HLA-A*02:01. In some embodiments of the invention, the T cell receptor recognizes a polypeptide epitope of human prostatic acid phosphatase selected from: ILLWQPIPV (SEQ ID NO: 47), TLMSAMTNL (SEQ ID NO: 48), IRSTDVDRTL (SEQ ID NO: 52), IMYSAHDTTV (SEQ ID NO: 55), KVYDPLYCESV (SEQ ID NO: 58), LLLARAASLSL (SEQ ID NO: 59), and LLFFWLDRSVLA (SEQ ID NO: 62). In certain embodiments of these compositions, the polynucleotide encodes amino acids of a TCR variable region and the vector comprises vector polynucleotides encoding a TCR constant region fused in frame with the TCR variable region (see, e.g. U.S. Patent Publication Nos. 20220354889, 20200138865, 20210363245 and 20210155941; and Coren et al., Biotechniques. 2015 Mar. 1; 58 (3): 135-9 (which describes aspects of the MSGV Hu Acceptor vector sold by Addgene™). Typically in these composition, the polynucleotide is disposed in a cell (e.g. a human CD8″ T cell). Optionally, for example, the polynucleotide is disposed in a CD8″ T cell is obtained from an individual diagnosed with a cancer that expresses a human prostatic acid phosphatase antigen (e.g. a prostate cancer); and the CD8″T cell is transduced with a vector comprising a polynucleotide encoding a TCR Vα polypeptide in combination with a polynucleotide encoding a TCR VB polypeptide such that a heterologous TCR is expressed on a surface of the CD8″ T cell, wherein the heterologous TCR recognizes a human prostatic acid phosphatase peptide associated with a human leukocyte antigen expressed on the surface of cells of the cancer.

In certain compositions of the invention, the polynucleotide encodes a segment of at least 5, 10, 25, 50 or 100 amino acids of a TCR polypeptide embodiment of the invention shown in Table A or Table B below (e.g., at least 5 or 10 amino acids present in an Alpha CDR1 polypeptide sequence, an Alpha CDR2 polypeptide sequence, an Alpha CDR3 polypeptide sequence, a Beta CDR1 polypeptide sequence, a Beta CDR2 polypeptide sequence or a Beta CDR3 polypeptide sequence). In certain compositions of the invention, the polynucleotide encodes a segment of at least 100 amino acids having an at least 98% sequence identity to amino acids encoded by SEQ ID NO: 1-SEQ ID NO: 42. In some embodiments of the invention, the T cell receptor (TCR) alpha chain polypeptide and/or the TCR beta chain polypeptide encoded by the polynucleotide comprises an amino acid substitution mutation of the wild type TCR amino acid sequence (e.g. SEQ ID NO: 1-SEQ ID NO: 42) that is selected to optimize its interaction with its cognate ligand (see, e.g. Sibener et al., Cell 174, 672-687, Jul. 26, 2018; and Zhao et al., Science 376, 155 (2022), the contents of which are incorporated herein by reference). In illustrative examples of such mutants, a polynucleotide encodes a segment of at least 5, 10, 25, 50 or 100 amino acids encoded by SEQ ID NO: 115-SEQ ID NO: 138.

Embodiments of the invention include methods of killing a cancer cells that express a human prostatic acid phosphatase peptide associated with a human leukocyte antigen expressed on the surface of cells of the cancer. For example, embodiments of the invention include methods of inhibiting growth of a prostate cancer cell comprising combining the prostate cancer cell with a CD8″ T cell transduced with a polynucleotide encoding a T cell receptor (TCR) alpha chain polypeptide and a TCR beta chain polypeptide; wherein when transduced into and expressed in the CD8″ T cell, the alpha chain polypeptide and the TCR beta chain polypeptide can form a T cell receptor that recognizes a polypeptide epitope on human prostatic acid phosphatase (PAP) expressed on the prostate cancer cell, thereby inhibiting growth of the prostate cancer cell. In certain of these embodiments, the T cell receptor recognizes a polypeptide epitope of human prostatic acid phosphatase in combination with human leukocyte antigen HLA-A*02:01. In some embodiments of the invention, the T cell receptor recognizes a polypeptide epitope of human prostatic acid phosphatase selected from: KELKFVTL (SEQ ID NO: 43), FQKRLHPYK (SEQ ID NO: 44), LSGLHGQDL (SEQ ID NO: 45), FQKRLHPYK (SEQ ID NO: 46), ILLWQPIPV (SEQ ID NO: 47), TLMSAMTNL (SEQ ID NO: 48), VLAKELKFV (SEQ ID NO: 49), MEQHYELGEY (SEQ ID NO: 50), GEYFVEMYYR (SEQ ID NO: 51), IRSTDVDRTL (SEQ ID NO: 52), IWSKVYDPLY (SEQ ID NO: 53), SVHNFTLPSW (SEQ ID NO: 54), IMYSAHDTTV (SEQ ID NO: 55), DFIATLGKLSG (SEQ ID NO: 56), DVYNGLLPPYA (SEQ ID NO: 57), KVYDPLYCESV (SEQ ID NO: 58), LLLARAASLSL (SEQ ID NO: 59), SPIDTFPTDPIK (SEQ ID NO: 60), WQPIPVHTVPLS (SEQ ID NO: 61), LLFFWLDRSVLA (SEQ ID NO: 62), YSAHDTTVSGLQM (SEQ ID NO: 63), YSAHDTTVSGLQMA (SEQ ID NO: 64), LSELSLLSLYGIHK (SEQ ID NO: 65), IATLGKLSGLHGQD (SEQ ID NO: 66), KELKFVTLVFRHGD (SEQ ID NO: 67), and IATLGKLSGLHGQDL (SEQ ID NO: 68). In certain embodiments of these methods, CD8⁺ T cells are combined in vivo so as to treat an individual suffering from prostate cancer. Optionally in these methods, the polynucleotide the polynucleotide encodes a segment of at least 100 amino acids having an at least 98% sequence identity to amino acids encoded by SEQ ID NO: 1-SEQ ID NO: 42 (as is known in the art, sequence identity is the ratio of the number of identical amino acids between the 2 aligned sequences/segments over the aligned length, expressed as a percentage).

Embodiments of the invention include methods of assessing a patient immune response to a prostate cancer or prostate cancer vaccination. Typically these methods comprise observing the induction or activation of T cells obtained from a patient having a prostate cancer or prostate cancer vaccination, wherein the induction or activation of T cells is observed in response to the T cell's exposure to a polypeptide epitope present on human prostatic acid phosphatase (PAP); and an observed induction or activation of T cells provides evidence of patient immune response to prostate cancer or prostate cancer vaccination. Optionally in these methods, T cells express a T cell receptor that recognizes a polypeptide epitope of human prostatic acid phosphatase selected from: ILLWQPIPV (SEQ ID NO: 47), TLMSAMTNL (SEQ ID NO: 48), IRSTDVDRTL (SEQ ID NO: 52), IMYSAHDTTV (SEQ ID NO: 55), KVYDPLYCESV (SEQ ID NO: 58), LLLARAASLSL (SEQ ID NO: 59), and LLFFWLDRSVLA (SEQ ID NO: 62).

Embodiments of the invention encompass methods of treating a disease or condition characterized by the expression of PAP. The treatment methodology comprises comprising administering an effective amount of a pharmaceutical composition comprising the modified T cell described herein to a subject in need thereof. The term “subject” is intended to include living organisms in which an immune response can be elicited (e.g., mammals). A “subject” or “patient”, as used therein, may be a human or non-human mammal. Non-human mammals include, for example, livestock and pets, such as ovine, bovine, porcine, canine, feline and murine mammals. Preferably, the subject is human. In typical embodiments of the invention, the human has a cancer expressing A PAP epitope recognized by a TCR. In some embodiments of the invention, the cells of the cancer form solid tumors. In illustrative embodiments of the invention, the cancer cells are prostate cancer cells.

A related embodiment of the invention includes a method for prophylaxis and/or therapy of an individual diagnosed with, suspected of having or at risk for developing or recurrence of a cancer, wherein the cancer comprises cancer cells which express A PAP epitope recognized by a TCR. This approach comprises administering to the individual modified human T cells comprising a recombinant polynucleotide encoding a TCR, wherein the T cells are capable of direct recognition of the cancer cells expressing the A PAP epitope recognized by a TCR, and wherein the direct recognition of the cancer cells comprises HLA class II-restricted binding of the TCR to the A PAP epitope recognized by a TCR expressed by the cancer cells.

With respect to use of the engineered CD8⁺ T cells of the present invention, the method generally comprises administering an effective amount (e.g. by intravenous or intraperitoneal injections) of a composition comprising the CD8⁺ T cells to an individual in need thereof. An appropriate pharmaceutical composition may be adapted for administration by any appropriate route, such as parenteral (including subcutaneous, intramuscular, or intravenous), enteral (including oral or rectal), inhalation or intranasal routes. Such compositions may be prepared by any method known in the art of pharmacy, for example by mixing the active ingredient with the carrier(s) or excipient(s) under sterile conditions.

In another aspect, the invention includes use of a polynucleotide or a modified CD8⁺ T cell described herein in the manufacture of a medicament for the treatment of a disease or condition characterized by the expression of PAP, in a subject in need thereof. In illustrative embodiments of the invention, the disease is a cancer expressing PAP epitope disclosed herein, for example, a prostate cancer.

The technology in this area is fairly developed and a number of methods and materials know in this art can be adapted for use with the invention disclosed herein. Such methods and materials are disclosed, for example in U.S. Patent Publication Nos. 20190247432, 20190119350, 20190002523, 20190002522, 20180371050, 20180057560, 20170029483, 20160024174, and 20150141347, the contents of which are incorporated by reference.

Certain aspects of the invention are disclosed in Mao et al. Proc Natl Acad Sci USA. 2022 Aug. 2; 119 (31): e2203410119. doi: 10.1073/pnas.220341011, the contents of both of which are incorporated herein by reference. All publications mentioned herein (e.g. those disclosed herein such as Zah et al., Nature Communications volume 11, Article number: 2283 (2020) and International Patent Applications PCT/US19/49484, WO 2021/046121 and PCT/US2020/037486, as well as Kantoff, Phillips, et al. “Sipuleucel-T Immunotherapy for Castration-Resistant Prostate Cancer.” N Engl J Med 2010; 363:411-422; Fong, Lawrence, et al. “Dendritic cell-based xenoantigen vaccination for prostate cancer immunotherapy.” J Immunol 2001; 167 (12) 7150-7156; and Nesterenko, Pavlo, et al. “Droplet-based mRNA sequencing of fixed and permeabilized cells by CLInt-seq allows for antigen-specific TCR cloning.” PNAS 2021; 118 (3)) are incorporated by reference to disclose and describe aspects, methods and/or materials in connection with the cited publications. Many of the techniques and procedures described or referenced herein are well understood and commonly employed by those skilled in the art. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

### EXAMPLES

### Example 1: Physical and in Silico Immunopeptidomic Profiling of a Cancer Antigen Prostatic Acid Phosphatase Reveals Targets Enabling TCR Isolation

Certain aspects of the invention disclosed below are found in Mao et al., Proc Natl Acad Sci USA. 2022 Aug. 2; 119 (31) (hereinafter “Mao et al.”), the contents of which are incorporated by reference.

Tissue-specific antigens can serve as targets for adoptive T-cell transfer-based cancer immunotherapy. Recognition of tumor by T cells is mediated by interaction between peptide-major histocompatibility complexes (pMHCs) and T cell receptors (TCRs). Revealing the identity of peptides bound to MHC is critical in discovering cognate TCRs and predicting potential toxicity. We performed multi-modal immunopeptidomic analyses for prostatic acid phosphatase (PAP), a well-recognized tissue antigen. Three physical methods including mild acid elution, co-immunoprecipitation, and secreted MHC precipitation, were used to capture a thorough signature of PAP on HLA-A*02:01. Twenty-seven PAP peptides in total were identified while only five of these peptides were predicted by the commonly used algorithm NetMHCpan 4.0. Peripheral blood mononuclear cells (PBMCs) from more than 20 healthy donors were screened with the PAP peptides. Twenty-one cognate TCRs against 7 distinct epitopes were identified using a single-cell isolation technique that detects intracellular IFNγ and TNFα. One TCR shows reactivity toward cell lines expressing both full-length PAP and HLA-A*02:01. Our results show that a combined multi-modal immunopeptidomic approach is productive in revealing target peptides and defining the first cloned TCR sequences for prostatic acid phosphatase.

### Prostatic Acid Phosphatase (PAP) is a Target for Prostate Cancer Immunotherapy

One way to prevent “on-target off-tumor” toxicity is to select tissue antigens expressed on non-essential organs. Patients with late-stage prostate cancers have often received a radical prostatectomy to remove the prostate gland (11). We chose prostatic acid phosphatase (PAP) among many previously defined prostate tissue antigens because: 1. The expression of PAP is restricted to the prostate and prostate cancer (12); 2. PAP expression can be found in >95% of prostate cancers (13); 3. Serum PAP elevation was found in >60% patients with relapsed prostate cancers (14); 4. The secreted form of PAP will not compete with TCR-PAP recognition, because the interaction is restricted to peptide bound to MHC I.

Previous efforts to target PAP led to the first FDA-approved cancer vaccine, sipuleucel-T (Provenge) (15). The clinical trials showed a median improvement in overall survival of 4.1 months in men with metastatic castration-resistant prostate cancer (15). T cell proliferation responses in vaccinated individuals were measured by a stimulation index (SI) (15). SI is defined as 3H-thymidine incorporation of T cells cultured with antigens divided by control groups (15). A positive T cell proliferation response was defined by SI>5 measured at week 6 post immunization (15). Among patients who received Provenge, 27.2% of patients displayed responses to PAP (8.0% in placebo) (15). Recent studies have also provided video evidence that T cells from sipuleucel-T treated patients are capable of lysing PAP″ target cells (16). Neither the PAP epitopes presented nor the cognate TCR sequences have been defined at the molecular level. Recovery of TCRs that specifically recognize PAP epitopes can lead to products for potential therapeutic treatments.

### In Silico Prediction of Epitopes on MHC I is an Important Strategy but not Sufficient

Multiple computational methods have been developed to predict peptide-MHC binding affinities with knowledge based on experimentally defined epitopes (17-19). In silico prediction can rapidly generate results and have been widely used (18). Previous efforts to identify PAP epitopes also mainly relied on motif-based prediction (20, 21). In a recent study, Wells et al assembled a consortium (TESLA) including 25 different prediction platforms for comparison (22). Only 6% of predicted peptides were found to be immunogenic by pMHC multimer staining (22). It is also difficult to find consensus among in silico pipelines. The overlap among different algorithms is limited in TESLA: only a median of 13% and maximum of 62% were found between any two randomly selected methods (22). Defining peptides bound to MHC I by prediction alone is challenging for two reasons: 1. Choosing the single best tool to use and 2. Assessing false positivity and false negativity of the prediction results.

### Using Physical Assays to Define the Immunopeptidome

An alternative way to define the immunopeptidome is to directly isolate peptides bound to MHC I and identify them by liquid chromatography and mass spectrometry (LC-MS). Multiple physical methods using mass spectrometry to define the immunopeptidome have been previously developed including mild acid elution (MAE), MHC co-immunoprecipitation (CoIP) and secreted MHC immunoprecipitation (sMHC-IP). MAE was one of the earliest approaches to isolate peptides from MHC I by using an isotonic acid buffer to destabilize peptide-MHC complexes (23). Although fast and convenient, this method can yield non-MHC bound peptides from other extracellular proteins. CoIP purifies peptide-MHC I complexes with monoclonal antibodies to generate results with less non-MHC peptides contamination (24, 25). This requires large quantities of antibody as well as expression of both the antigens of interest and the desired HLA types on target cells. The sMHC-IP technique requires the engineering and expression of soluble single-chain MHC in cell lines for affinity capture (26, 27). This protocol requires manipulation of cell lines and might generate peptides only presentable on artificial constructs. There is no consensus for the single best approach. To capture a more comprehensive immunopeptidomic signature of PAP, we combined all three above-mentioned approaches on HLA-A*02:01, one of the most common subtypes (28).

Identification of TCRs from Antigen-Reactive Single T Cells

To date, no single-cell MHC I-restricted TCR sequence has been defined and disclosed against PAP epitopes on the publicly available Immune Epitope Database (IEDB) (29). One of the major challenges has been to enrich and to identify cognate T cells for single-cell sequencing. We recently developed a technique, CLint-seq, to enable single-T cell isolation of activated cells. Cells were fixed with a disulfide bond-based reversible crosslinker (DSP) and sorted based on intracellular activation markers by Fluorescence-activated Cell Sorting (FACS) (30). Utilization of a reversible crosslinker allows the cells' mRNA to be released from mRNA-protein crosslinked complexes. These mRNAs can then be efficiently reverse transcribed and meet the quality that is compatible with 10× Genomics single cell TCR sequencing platform (30, 31). T cells stimulated by cognate peptides can produce cytokines such as IFNγ and TNFα, which can be trapped and intracellularly stained. Using physically determined PAP epitopes, 21 peptide-reactive TCRs were successfully isolated with CLint-seq from healthy donor PBMCs.

### Results:

### Multi-Modal Immunopeptidomic Profiling of PAP on HLA-A*02:01

Both physical and in silico approaches were used to define a thorough HLA-A*02:01 immunopeptidomic signature of PAP. A commonly used algorithm, NetMHCpan 4.0, was applied to profile PAP epitopes on HLA-A*02:01 (18). 40 PAP peptides were selected as potential good binders on HLA-A*02:01 using the top 2 percentile as a cutoff (Supp Table 1 in Mao et al.).

To determine the presence of these predicted peptides and others, three previously published physical methods were performed including mild acid elution (MAE), co-immunoprecipitation (CoIP), and secreted-MHC immunoprecipitation (sMHC-IP) (FIG. 1). The MAE protocol uses an acidic buffer (pH 3.3) to dissociate peptide-MHC I complexes. It was applied on both mono-allelic (K562-A2-PAP) and multi-allelic (M202-PAP) HLA-A*02:01⁺ cell lines. K562-A2-PAP is considered a mono-HLA-allele cell line because wild type K562 cells are deficient in surface MHC I (32). This strategy identified 11 PAP peptides in total (Supp FIG. 1a; Table 1 in Mao et al.).

Since the treatment with MAE can induce release of non-MHC peptides, an alternative approach, CoIP, was performed on the same two cell lines. This approach uses monoclonal antibody (clone W6/32) to enrich for MHC I released from cell surfaces after lysis (24, 25, 33). Peptides bound to MHC I are then dissociated from purified products and analyzed by LC-MS/MS. 12 PAP peptides were recovered by CoIP (Supp FIG. 1a; Table 1 in Mao et al.). 2 peptides overlapped with those found by MAE (Supp FIG. 1a; Table 1 in Mao et al.).

Secreted MHC-IP (sMHC-IP) was previously developed to enforce higher expression of an engineered soluble form of MHC I as single chain dimer (SCD) (26, 27). A recently published sMHC-IP platform, ARTEMIS, achieves robust expression and secretion of soluble HLA-A*02:01 molecules (27). This engineered form contains a hexa-histidine-tag (6× His tag) to increase enrichment efficacy by Ni-NTA agarose. 8 peptides were recovered in sMHC-IP including 6 not found in the other two physical methods (Supp FIG. 1a, Table 1 in Mao et al.). No single peptide was found by all three methods (Supp FIG. 1a; Table 1 in Mao et al.). Comparison between the physical approaches and in silico approaches shows only 5 peptides overlapped, which makes up 12.5% of total peptides predicted by NetMHCpan 4.0 (Supp FIG. 1b; Table 1 in Mao et al.).

BLAST analysis was then performed on all the physically recovered PAP epitopes against the human protein library to test their specificity to PAP (34). All 27 PAP peptides are unique to PAP sequences. Peptides with similar sequences mostly came from other members of the acid phosphatase family such as lysosomal acid phosphatase and testicular acid phosphatase (Supp Table 2 in Mao et al.).

### Evaluating HLA-A*02:01 Specificity of Recovered PAP Peptides

Some of the peptides recovered by physical methods might originate from non-HLA-A*02:01 subtypes or peptide fragments not on MHC I. To assess the HLA-A*02:01 specificity of recovered PAP epitopes, T2 cell binding assays were performed. The T2 cell line is deficient in the transporter associated with antigen processing (TAP) protein, which is responsible for loading peptides onto MHC I. As a result, only a limited amount of unstable MHC I (including HLA-A*02:01) molecules are naturally presented on the T2 cells (35). In T2 binding assays, chemically synthesized candidate peptides (>80% purity) are exogenously added into growth media. Epitopes with A*02:01 specificity can form stable peptide-MHC complexes and induce accumulation of those molecules. The quantity of HLA-A*02:01 can then be quantified by anti-A2 antibodies (clone BB7.2) conjugated with FITC using flow cytometers (FIG. 2a).

27 PAP peptides defined by physical methods were tested in T2 binding assays. 6 out of 27 PAP peptides show high HLA-A2 signal when exogenously pulsed on T2 cells (Table 1, FIG. 2b in Mao et al.). All 6 peptides can be detected by the sMHC-IP, including one epitope found by both sMHC-IP and CoIP. 5 out of these 6 peptides passed the 2% selection cutoff of NetMHCpan 4.0 as strong HLA-A*02:01 binders (Table 1 in Mao et al.).

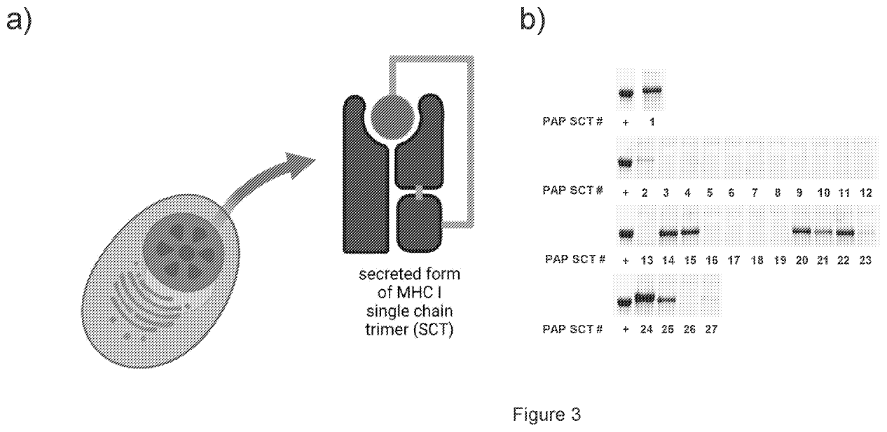

Peptide-MHC I complexes processed by endogenous machinery might show different stability compared to exogenous peptide pulsing (such as in T2 binding assays). This can be the result of post-translational modifications (PTMs). A recently developed technique, secreted single-chain trimer (SCT), was used to evaluate relative stability of pMHC of interest (36). In the secreted SCT construct, MHC I heavy chain (HLA-A*02:01 alpha chain with H74L and Y84C mutation), light chain (beta-microglobulin), and corresponding peptide were tethered by linkers as one single chain molecule (FIG. 3a). Constructs were expressed in cells and released into culture supernatant. Only peptides favored by HLA-A*02:01 are expected to generate a stable SCT and have higher yields. The quantity of SCTs was measured by band intensity on SDS-PAGE and normalized against a well-known WT1 cancer epitope that is restricted on HLA-A*02:01 (RMFPNAPYL) (FIG. 3b) (37, 38).

9 peptides were found to have relatively high stability on HLA-A*02:01 when using a cutoff of 0.2 normalized to the control WT1 epitope (Table 1; FIG. 3b in Mao et al.). 5 out of these 9 PAP peptides were not among the selected candidates by NetMHCpan 4.0 (top 2%) (Table 1 in Mao et al.). Only 4 out of these 9 peptides score positively in the T2 binding assay, suggesting that each evaluation technique may address different aspects of peptide-MHC stability (Table 1 in Mao et al.). Notably, PAP A2_24 shows a higher yield (1.40) than the positive control, but it has a poor predicted score by NetMHCpan 4.0 (24.22%) (Table 1; FIG. 3b in Mao et al.).

### A Post-Translationally Modified PAP Peptide Shows Increased Binding Affinity to HLA-A*02:01

PAP-A2-24 shows contradictory results of HLA-A*02:01 binding in different stability assays. One possible explanation is that PAP-A2-24 has been post-translationally modified. Previous literature reports N-glycosylation on the asparagine of PAP-A2-24 (N220 of PAP) (39). To investigate if the N-glycosylated form of PAP-A2-24 is presented, SCT products of both PAP-A2-24 (SVHNFTLPSW (SEQ ID NO: 54)) and PAP-A2-25 (IMYSAHDTTV (SEQ ID NO: 55)) were treated with PNGase F, which can specifically remove N-glycan (40). SDS-PAGE results indicate that PAP-A2-24 SCT showed a band of apparent higher molecular weight than PAP-A2-25 prior to PNGase F treatment. Both SCTs migrate similar distances in the gel after de-glycosylation (Supp FIG. 2a in Mao et al.). This result indicates that an additional N-glycan exists on PAP-A2-24 in addition to glycosylation sites on the MHC I heavy and light chains. The only sequence difference between PAP-A2-24 SCT and PAP-A2-25 SCT resides in their peptide fragments. It is very likely the additional N-glycan was within the peptide (SVHNFTLPSW (SEQ ID NO: 54)).

Spectrums were also re-analyzed to confirm if the de-glycosylated form of PAP-A2-24 also exists. Previous reports suggests that N-glycosylated asparagine (N) can undergo enzymatic deamidation to aspartate (D) (41). Both forms were detected by LC-MS in CoIP results: SVHNFTLPSW (SEQ ID NO: 54) and SVHDFTLPSW (SEQ ID NO: 69) (Supp FIG. 2b in Mao et al.).

Isolation of PAP Peptide Specific TCRs from Healthy Individuals' PBMCs

PBMC cells collected from multiple commercially available normal donors (n>20) were screened to find TCRs reactive to PAP peptides. 27 chemically synthesized peptides were added to total PBMCs, which contain a mixture of antigen presenting cells (e.g. monocytes and B cells) that are able to prime T cells. The T cells were then allowed to culture and expand for 10 days in culture. The CLint-seq protocol was then applied on those stimulated cells to isolate reactive candidate T cells (30). As discussed above, TNFα⁺/IFNγ⁺ fixed CD8 T cell population was sorted by FACS to enrich for the reactive population. TCR pairs appearing more than once in 10× Genomics sequencing results (Frequency >1) were selected as potential PAP-reactive clones. 124 candidate a/B pairs were recovered from 8 healthy individuals, including 3 females, 4 males and 1 unknown (Supp Table 3 in Mao et al.).

To determine whether these TCRs are reactive against PAP, TCR variable regions of both alpha and beta chains from all selected candidates were then synthesized into a DNA fragment for cloning. Constant regions of both alpha chain and beta chain (TRAC and TRBC) were replaced by mouse constant regions to decrease mispairing with endogenous human TCRs. Paired TCR alpha chain and beta chain were linked with a mutated self-cleaving 2A peptide linker (F2Aopt) to ensure equal expression (42).

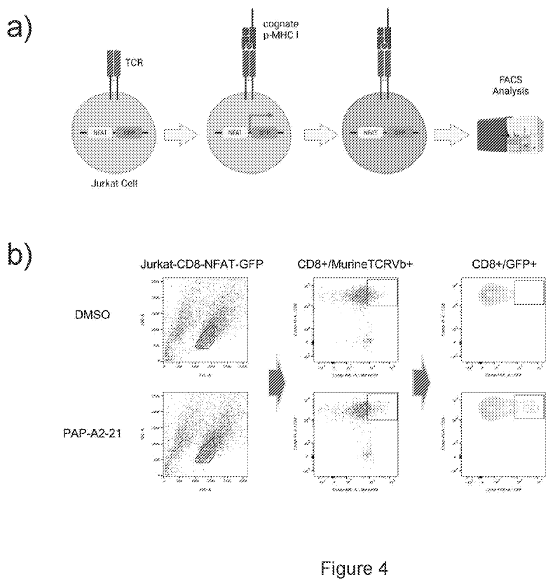

Engineered TCR sequences were then cloned into the pMAX-Cloning vector for rapid functional screening using electroporation. pMAX constructs containing a TCR of interest were electroporated into the Jurkat-CD8-NFAT-GFP cell line, which is used as a reporter system. In Jurkat-CD8-NFAT-GFP cells, GFP expression is induced by the binding and activation of NFAT promoter repeats after TCR activation (FIG. 4a). GFP expression can then be quantified by flow cytometry to determine if a TCR recognized cognate peptide-MHC I. Murine TCR beta chain was measured by FACS to estimate transfection efficiency. K562 cells were transduced with HLA-A*02:01-IRES-GFP (K562-A2) by lentivirus and used as target cells during the test (Methods). Individual chemically synthesized PAP peptides were added into and presented by K562-A2 cells. Effector cells (Jurkat) and target cells (K562) were mixed at a ratio of 2:1. From 124 candidate clones, 21 TCRs were found to recognize 7 distinct PAP peptides defined previously by LC-MS (Table 1; Supp Table 4 in Mao et al.). These 21 TCRs were from 3 individuals included 2 males and 1 female (Supp Table 3,4 in Mao et al.).

### Functional Validation of Candidate TCRs in Human PBMCs

21 candidate TCRs which showed reactivity in the Jurkat-CD8-NFAT-GFP system were then tested in human PBMC cells. The selected TCR constructs with mouse constant regions were followed by truncated low-affinity nerve growth factor receptor (delta LNGFR) as a transduction marker. Candidate TCRs were transduced into human PBMCs with the pMSGV retroviral system (9) (Methods). Surface dLNGFR level was measured by FACS to estimate efficiency of transduction. Murine TCR beta chain was also quantified by FACS to assess if TCRs traffic to the cell surface. Tetramers that contain individual PAP peptide of interest were produced and used on engineered PBMC to ensure specific recognition (Supp FIG. 3 in Mao et al.)

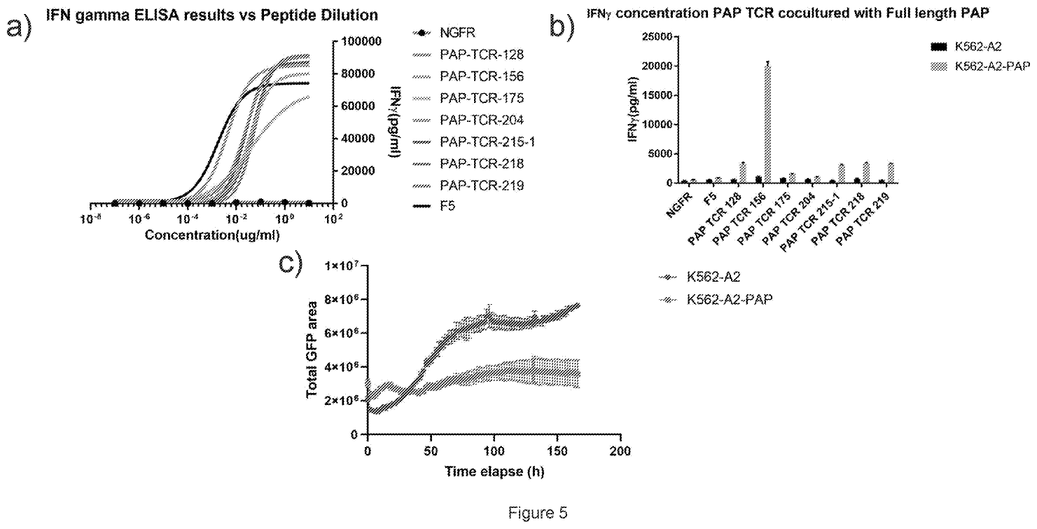

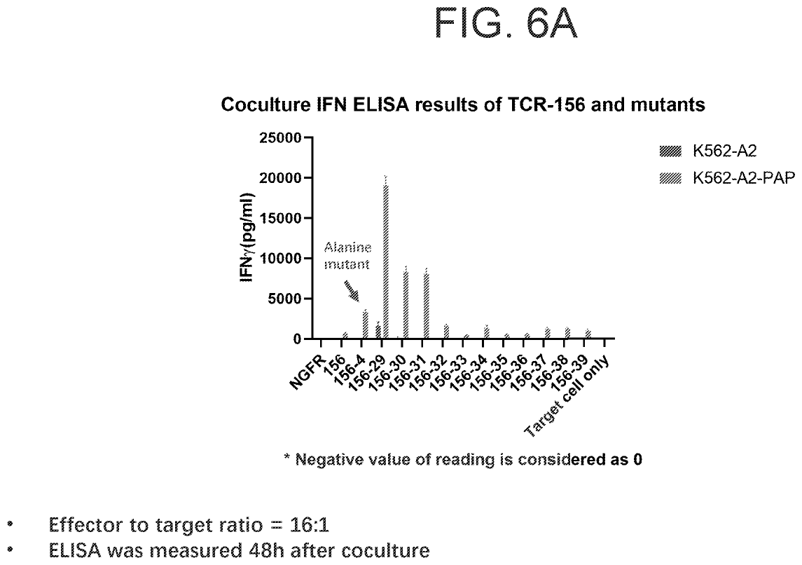

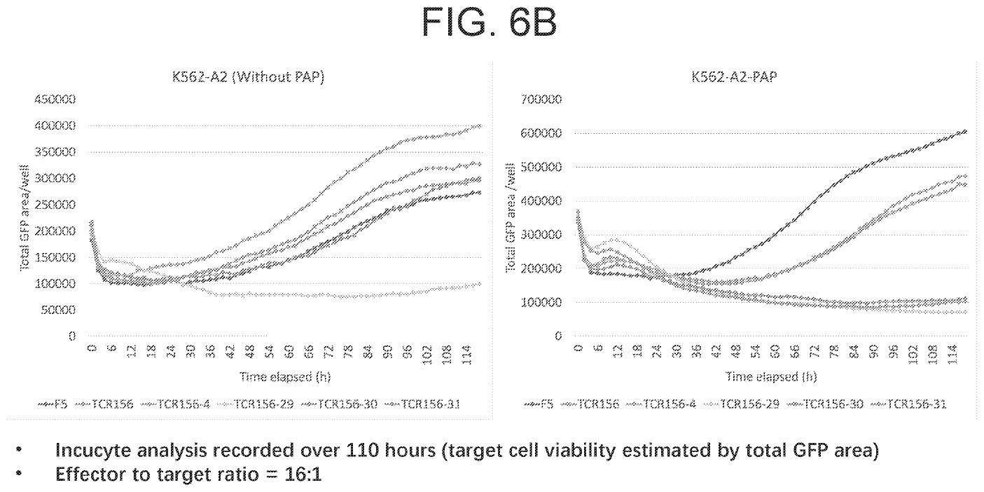

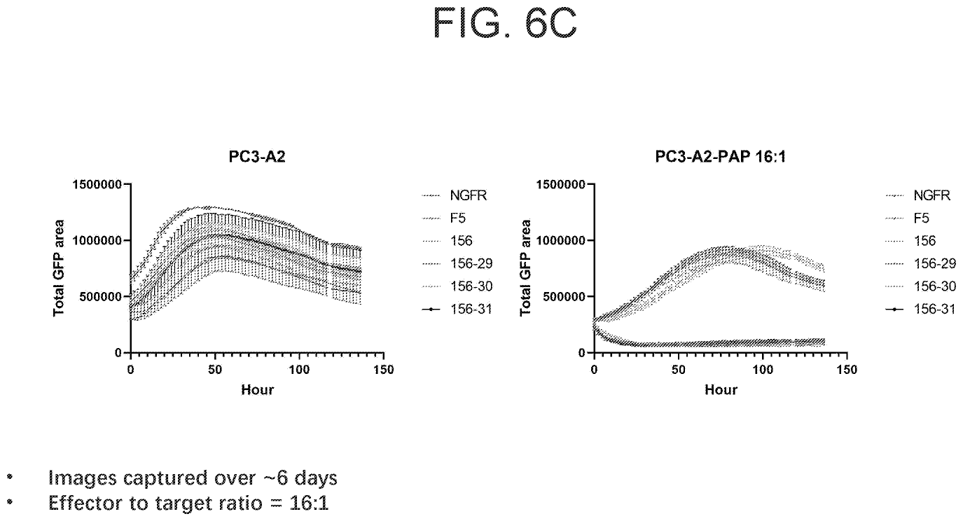

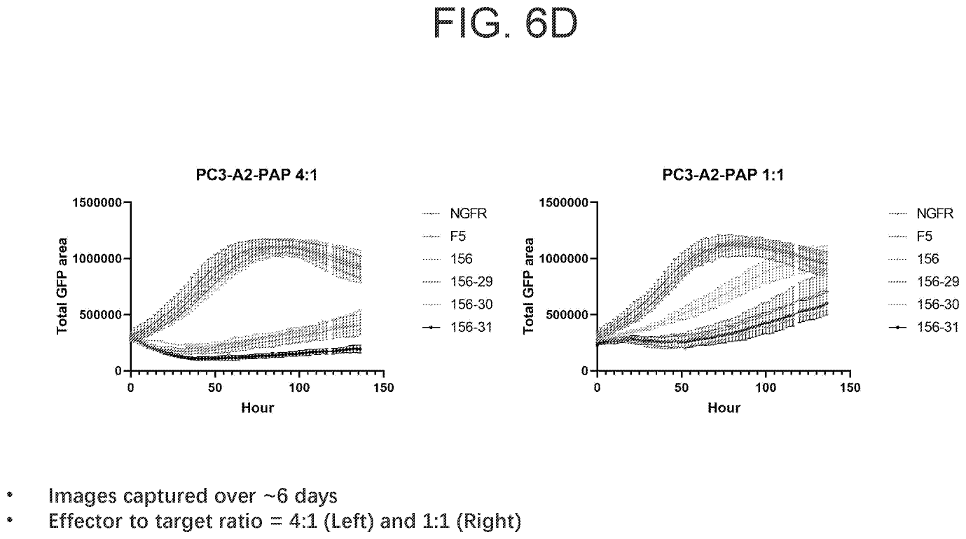

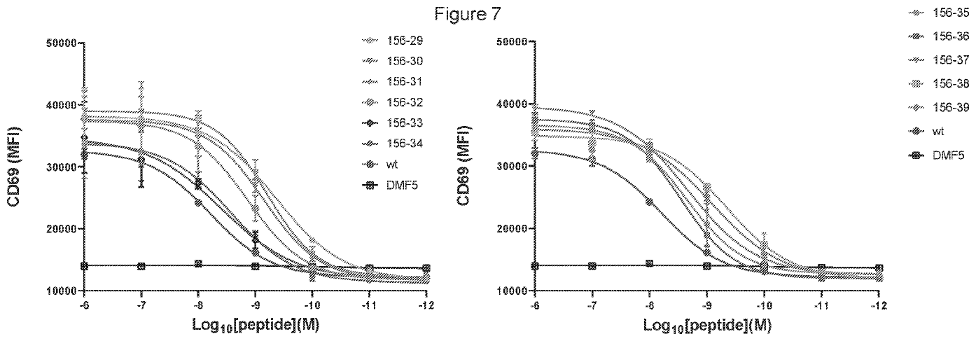

Stimulated T cells that recognize cognate peptide bound to MHC I can release cytokines such as IFNγ. ELISA was performed to quantify released IFNγ by using recombined IFNγ as a standard (Methods). Individual PAP peptides were added exogenously onto K562-A2 cells. Engineered PBMCs and target K562-A2 cells were mixed at a ratio of 2:1 (effector: target). The supernatants of the coculture experiments were then collected after 48 hours. 7 TCRs showed significant IFNγ signal against 3 distinct PAP peptides when expressed in human PBMCs (FIG. 5a; Table 1 in Mao et al.). Notably, 5 out of these 7 TCRs are against a peptide (PAP-21) that did not pass the prediction cutoff (<2%) using the NetMHCpan 4.0 algorithm (Table 1 in Mao et al.).

TCRs displaying high IFNγ signal in PBMCs were tested with serial dilution of cognate peptides to compare their relative potency with a clinically tested TCR, F5. This TCR was previously isolated from a melanoma patient against a MARTI epitope (EAAGIGILTV) (6). F5 TCR can induce tumor regression in patients without affinity maturation to increase its potency and served as a control in our experiments (6). Chemically synthesized peptides were tested at various concentrations on K562-A2. PBMCs expressing candidate PAP TCRs were mixed at a ratio of 2:1 (effector: target). IFNγ ELISA was performed on the collected supernatant after 48 hours. Notably, one PAP TCR (PAP-TCR-204) shows a similar level of activation compared to F5 by peptide dilutions, while the remaining six TCRs showed weaker results (FIG. 5a).

PBMCs expressing these 7 TCRs were then cocultured with target cells expressing full length PAP to test their ability for recognizing processed PAP epitopes. Full length PAP isoform 2 (TM-PAP) was transduced into the K562-A2 cell line by lentivirus. The transduced population was single cell sorted and expanded to create clonal cell lines that have strong expression of PAP. TCR-engineered PBMCs were mixed with target K562-A2-PAP cells at a ratio of 16:1 (effector: target). The F5 TCR and dLNGFR only (without TCR) empty vector transduced PBMCs were used as negative controls. ELISA was performed on coculture supernatant after 48 hours. One TCR (PAP-TCR-156) showed specific full-length PAP recognition with IFNγ produced at 20,000 pg/ml (FIG. 5b). Four TCRs (128, 215-1, 218 and 219) produced low IFNγ signal (around 3,000 μg/ml) (FIG. 5b).

Cytotoxicity of the candidate PAP TCRs was assessed by recording total live target cells using the IncuCyte platform. Target K562-A2-PAP cells co-express GFP and can be distinguished from GFP PBMC cells by real-time imaging and analysis. Live cell imaging was taken every two hours to record number of target cells over a time course of 120 hours. GFP signals were then processed by IncuCyte analysis tool to estimate the area of target cells. One of the candidate TCRs, PAP-TCR-156, is able to inhibit growth of cells expressing full-length PAP (FIG. 5c). Total GFP area of K562-A2-PAP is maintained at similar level during the 150-hour co-culture with PBMCs expressing PAP-TCR-156 (FIG. 5c). Total GFP area for K562-A2 cells showed a threefold increase as a negative control (FIG. 5c).

### Materials and Methods

Mild acid elution: Mild acid elution protocol to elute MHC I-associated peptides mainly based on previously published protocol with a few changes (53). 1-2×10⁸ cells were used. M202-PAP cells were dissociated with 1×PBS+1 mM EDTA, while K562-A2-PAP cells were collected by spinning down at 1500 RPM with 5 mins. Target cells were then washed 3 times with 1× HBSS buffer (Thermo Fisher). 25 ml mild acid elution buffer (0.131M citric acid, 0.066M Na₂HPO₄, 150 mM NaCl, 0.3 uM Aprotinin, 5 mM Iodoacetamide, pH=3.3) was applied to target cells and gently rocked for 2 mins under room temperature. Samples were then spin at 4000×g for 5 mins at 4 degree Celsius and supernatant was harvested. Formic acid was added to the samples to reach a final concentration of 0.1% (v/v). 3 ml C18 solid phase extraction cartridge (3M) was pre-rinsed by 99.9% acetonitrile (ACN)+0.1% formic acid for 3 times. MAE samples were then added to the C18 column followed by 3 times washing of 0.1% formic acid in water. C18 column was then eluted with 200 ul of 40% ACN+5% formic acid+55% H₂O for 3 times. Samples were then passed through 3kd centrifugal filters (Millipore) for 90 mins at 4000×g at 4 degrees Celsius. Flow-through was then dried by vacuum centrifugation and stored in −20° C. until MS analysis.

MHC I CoIP: CoIP protocol was modified based on previous published procedures (54, 55). 1-2×10⁸ M202-PAP or K562-A2 PAP cells were collected either by non-enzymatic dissociation reagents (1×PBS+1 mM EDTA) or by spinning down with 1500 rpm for 5 mins. Cells were first washed 3 times with 1×PBS. Cells were then lysed with CoIP lysis buffer (20 mM Tris (pH8.0), 1 mM EDTA, 100 mM NaCl, 1% Triton X-100, 60 mM n-octylglucoside, 1 mM PMSF (Sigma-Aldrich), protease inhibitor (Roche Life Science) and 1 mg/ml DNase I (Roche Life Science) with 1 ml lysis buffer per 10⁷ cells. Samples were then rocked for 1 hour at 4° C. Lysates were then centrifuge at 10000×g for 20 mins to pellet debris. Supernatant were then combined with GammaBind Plus Sepharose beads (GE Lifesciences) that have been conjugated with W6/32 antibodies (BioXCell) at the ratio of 1 ml beads per 10⁸ cells. Mixture of beads and lysates were rocked at 4° C. for 180 mins. Mixture was then loaded on to Poly-Prep Chromotography Column (Bio-Rad). Column was then washed 4 times with 10 ml wash buffer I (CoIP wash buffer I: 20 mM Tris (pH8.0), 1 mM EDTA, 100 mM NaCl, 60 mM n-octylglucoside and 1 mg/ml DNase I), 4 times with 10 ml wash buffer II (CoIP wash buffer II: 10 mM Tris (pH8.0)), and 1 time with 10 ml Ultrapure H₂O (Thermo Fisher). Peptides were released from beads by adding 10% Acetic Acid (Sigma) for 2 mins and cleaned up by spinning 30 secs at 3000×g with 0.45 μm Costar Spin-X centrifuge tube filters (Corning). Samples were then snap frozen and stored at −70° C. until further processing.

Secreted MHC-IP with ARTEMIS protocol: ARTEMIS protocol was based on previously published protocol (27). Expression of both secreted form of HLA-A2 and PAP was achieved by using lentiviral transduction system in free style 293-F cells (Thermo Fisher). 400 ml supernatant containing secreted MHC I was purified by Ni-NTA agarose (1 μL slurry per 1 ml supernatant). Slurry was loaded and wash in Poly-Prep Chromatography column. Samples after denaturation were stored in −70° C. until further processing.

### Lc-Ms Analysis:

Eluted samples were loaded to HyperSep C18 Column (Thermo Scientific 60108-390) and washed 3 times with 0.1% formic acid, then eluted with elution buffer (40% Acetonitrile, 0.1% formic acid). Desalted samples were lyophilized by speed vacuum and then reconstituted in the water. Next, samples were processed with detergent removal kit to remove residual detergent from the lysis buffer. Finally, acidified the samples to contain 5% formic acid before loaded to LC-MS. Samples were delivered to Orbitrap Fusion Lumos hybrid mass spectrometer by a 140-min gradient (0-5-min, 1-5.5% B, 5-128 min, 5.5-27.5% B, 128-135 min, 27.5-35% B, 135-136 min, 35-80%. B, 136-138 min, 80% B, 138-138.5 min, 80-1% B, 138.5-140 min, 1% B, B: 80% CAN+0.1% formic acid). The acquisition was conducted under data-dependent acquisition (DDA) mode: the full MS scan was acquired under 120K resolution in the Orbitrap mass analyzer, and singly charged ions with >800m/z and multi-charged ions were selected to be fragmented with High-energy Collision Dissociation (HCD) at 32% collision energy and then performed MS/MS scan under 15K resolution in Orbitrap. Dynamic exclusion was enabled to not repeat selecting ions with same m/z in 60 seconds. Database search was performed using Crux pipeline (v3.2) against EMBL human reference proteome (UP000005640human_9606), with non-specific digestion, PSM and peptide FDR is set to 1% threshold.

T2 peptide binding assay: T2 cells (ATCC) were cultured in IMDM (Thermo Fisher) with 20% FBS (Omega Scientific). Before peptide loading, 2×10⁵ cells were resuspended in 100 ul of serum free RPMI (Thermo Fisher) and added into each well of 96 U-bottom tissue culture plates (Corning). Chemically synthesized peptides were diluted into multiple concentration with serum free RPMI and added into designated well with T2 cells. Cells with peptides were co-cultured overnight in incubator at 37° C. Cells were then washed 2 times with 1×PBS and stained with 2 ul per well anti-HLA-A2 FITC antibodies (clone BB7.2, Biolegend). Quantity of HLA-A2 molecules were quantified by FACS.

SCT quantification assay: SCT constructs (mutant H74L/Y84C) with individual PAP peptides were synthesized according to the previously published protocol (36).

Cell culture: K562 (ATCC), M202 (gift from A. Ribas at UCLA) and Jurkat-NFAT-ZsGreen (gift from D. Baltimore at Caltech) were cultured in RPMI 1640 (Thermo Fisher) with 10% FBS (Omega Scientific) and Glutamine (Fisher Scientific). 293T (ATCC) was cultured in DMEM (Thermo Fisher) with 10% FBS and glutamine. Naïve Peripheral Blood Mononuclear Cells (PBMCs) for stimulation were cultured in TCRPMI with 50U/ml IL-2 (Peprotech) and chemically synthesized PAP peptides of interest (>80% purity, Elim Biopharm) as previously described (P Nesterenko, Cell Reports, 2021). TCRPMI media includes: RPMI 1640 (Thermo Fisher), 10% FBS (Omega Scientific), Glutamax (Thermo Fisher), 10 mM HEPES (Thermo Fisher), non-essential amino acids (Thermo Fisher), sodium pyruvate (Thermo Fisher) and 50 uM β-mercaptoethanol (Sigma). PBMCs for retroviral transduction were first activated by CD3/CD28 dynabeads (Thermo Fisher) and cultured in T cell media (TCM): AIM V media (Thermo Fisher), 5% Human AB serum (Omega Scientific), 50 U/ml IL-2 (Peprotech), 0.5 ng/ml IL-15 (Peprotech), Glutamax (Thermo Fisher) and 50 μM β-mercaptoethanol (Sigma).

CLInt-seq: Isolation of reactive T cells by CLInt-seq was performed on stimulated PBMCs according to previously published protocol (30). After 7-10 days coculture with the PAP peptide pool, PBMCs were transferred into 96 well U plate and rested overnight. Cells were then cultured with 10 ug/ml peptide pool and 1 ug/ml CD28/49d antibodies (BD Biosciences) for 1 hour before adding Brefeldin A (Biolegend). After about 8 hour incubation at 37° C., cells were treated as previously described and stained for CD3⁺/CD4⁻/CD8⁺/TNFα⁺/IFNγ⁺ population by FACS (56).

Single-cell TCR sequencing: CD8⁺ T cells that produce both TNFα and IFNγ were sorted into ˜30 ul of 0.04% BSA solution. If fewer than 1000 cells were isolated, 5000-10000 K562 cells would be sorted into the same tube as carrier population. 10× Genomics' single-cell TCR V (D) J library was then constructed by the UCLA Technology Center for Genomics & Bioinformatics. TCR pairs were then sequenced on MiSeq (Illumina).

Jurkat-NFAT-GFP essay: Candidate TCRs were rapidly screened in Jurkat-NFAT-GFP cells as described previously (56).

Transduction of TCRs in PBMC: Engineering of candidate TCRs in PBMC were performed according to previous publication (56).

Preparation of MHC Tetramers: MHC tetramers used to stain candidate PAP TCRs were synthesized and prepared according to a previously published protocol (57).

T cell activation analysis: For peptide pulsing co-culture experiments, target cells were mixed with TCR engineered PBMCs at a ratio of 1:2 (T: E) in the media desired by target cells and supplemented with 1 μg/ml of anti-CD28/CD49d antibodies (BD Biosciences). For cell lines expressing full-length PAP, target cells were first treated by 2 ng/ml IFNγ and 3 ng/ml TNFα for 8-10 hours. Target cells and PBMCs were then mixed at a ratio of 1:16 (T: E) for co-culture analysis. Supernatants were collected after 48 hours and analyzed by ELISA (BD Biosciences) to estimate IFNγ concentration.

Cytotoxicity analysis by IncuCyte: Target cells were plated onto 96 well tissue culture plates coated with 0.001% poly-L-lysine (Sigma) and kept in 37° C. for ˜2 hours. TCR-engineered PBMCs were then added to desired wells with effector: target ratio of 2:1 (peptide pulsed target cells) or 16:1 (full length PAP target cells). Plates with cell mixtures were analyzed by the IncuCyte system for 120 hours using GFP surface area to estimate killing of T cells.

### DISCUSSION

Our study using multiple immunopeptidomic approaches reveals 27 potentially HLA-A*02:01-restricted PAP peptides. We were able to recover 21 candidate PAP TCRs against 7 of the defined epitopes. 7 TCRs show reactivity against 3 distinct epitopes on peptide-pulsed target cells when engineered into human PBMC cells. Among them, one TCR (PAP-TCR-156) can recognize peptides presented on cell lines expressing full length PAP and HLA-A*02:01 allele.

All three physical assays (MAE, CoIP and sMHC-IP) were able to generate immunogenic peptides. For TCRs that work efficiently in PBMCs, all 3 cognate peptides can be detected by sMHC-IP. sMHC-IP seems to be the most efficient method to recover immunogenic epitopes, despite our relatively small sample size.

Epitopes defined by physical methods can be used to develop reagents for PAP-specific T cells. Peptides of interest can be refolded into MHC-based multimers as detection and isolation reagents. The common form is called “tetramers,” in which four peptide-MHC molecules are attached on a streptavidin molecule. More complexed versions of multimers were also available by adding more fluorochromes and increasing number of MHC monomers such as pentamers or dextramers (43). Production of MHC multimers relies mainly on knowing the identities of peptides. 7 of our recovered PAP TCRs against 3 distinct PAP peptides can be specifically stained by their cognate tetramers. Other candidates in our list may also be used in making multimers. These reagents can be useful in prescreening patients who have been treated with Provenge or healthy donors for PAP-reactive T cells.

One of the PAP epitopes (PAP-A2-24) shows altered affinity toward HLA-A*02:01 after glycosylation. Both the native form and de-glycosylated form (N to D) were detected by LC-MS. Post-translational modification such as glycosylation can potentially generate a larger pool of epitopes for immunotherapies, since cancers can generate abnormal carbohydrate modification on proteins (44, 45).

One of our candidate TCRs, PAP-TCR-156, shows the potential of recognizing cell lines expressing full-length PAP. The credentialing of this TCR shows weak T cell response by IFNγ and the cytotoxicity assay. Increasing the potency of these candidate TCRs is needed for future applications and tests.

One way to enhance a T cell's sensitivity and potency is to increase the affinity of its TCRs, a process called “TCR affinity maturation” (46). Previous results have demonstrated that higher affinity can lead to faster and stronger responses (47). Common methods for TCR affinity maturation include 1. Untargeted mutagenesis, 2. Site-directed mutagenesis, and 3. Single Amino acid (AA) screening of TCR complementarity-determining regions (CDRs) (7, 48, 49).

Using alternative sources of T cells may also provide us with high potency TCRs. PBMCs from healthy donors were used as our source of T cells. TCRs against tissue antigens, such as PAP, may have been deleted during thymic negative selection (50). T cells from a thymus-free in vitro culture may serve as a better source, since these T cells do not undergo negative selection (51, 52). Querying our defined PAP epitopes against T cells from these alternative sources might provide TCRs with higher affinities and specificities.

The PAP-specific TCRs we defined can serve as a starting point for in vivo experiments and potential clinical development. The PAP epitope information gathered can be used to create detection and capture reagents. It is appreciated that future engineering and improvement need to be performed to increase candidate TCRs' potency. T cells from alternative sources can also be used to increase the diversity of our candidate TCR pool.

### Table a: Illustrative Pap TCR Embodiments of the Invention

Embodiments of the invention include compositions of matter comprising a polynucleotide encoding a TCR polynucleotide (e.g. a TCR polynucleotide disposed in a vector). In typical embodiments, the polynucleotide encodes a Vα T cell receptor polypeptide and/or a Vβ T cell receptor polypeptide; and is disposed in the vector such that when a Vα/Vβ T cell receptor comprising the Vα T cell receptor polypeptide and/or the VB T cell receptor polypeptide is expressed in a CD 8⁺ T cell, the Vα/Vβ T cell receptor expressed by the CD 8⁺ T cell recognizes/targets a prostatic acid phosphatase peptide associated with a human leukocyte antigen (e.g., HLA-A*02:01).

Twenty one illustrative working TCR embodiments of the invention are disclosed below.

1. PAP-TCR-128
A. Target Peptide Sequence:
(SEQ ID NO: 59)
LLLARAASLSL (“PAP_A2_21”).
B. Valpha CDR3 Region Polypeptide Sequence:
(SEQ ID NO: 70)
CAASVDEKLTF
C. Vbeta V Region Polypeptide Sequence:
(SEQ ID NO: 71)
CASSMYNEQFF
D. Alpha Chain V(D)J Region Polynucleotide Sequence:
(SEQ ID NO: 1)
Gaccagcaagttaagcaaaattcaccatccctgagcgtccaggaaggaagaatttctattctgaactgtgactatactaaca
gcatgtttgattatttcctatggtacaaaaaataccctgctgaaggtcctacattcctgatatctataagttccattaagga
taaaaatgaagatggaagattcactgttttcttaaacaaaagtgccaagcacctctctctgcacattgtgccctcccagcct
ggagactctgcagtgtacttctgtgcagcaagcgtagatgagaaattaacctttgggactggaacaagactcaccatcatac
ccaat
E. Beta Chain V(D)J Region Polynucleotide Sequence:
(SEQ ID NO: 2)
Gatactggagtctcccagaaccccagacacaagatcacaaagaggggacagaatgtaactttcaggtgtgatccaatttctg
aacacaaccgcctttattggtaccgacagaccctggggcagggcccagagtttctgacttacttccagaatgaagctcaact
agaaaaatcaaggctgctcagtgatcggttctctgcagagaggcctaagggatotttctccaccttggagatccagcgcaca
gagcagggggactcggccatgtatctctgtgccagcagcatgtacaatgagcagttcttcgggccagggacacggctcaccg
tgctagagGACCT
2. PAP-TCR-131
A. Target Peptide Sequence:
(SEQ ID NO: 55)
IMYSAHDTTV (“PAP_A2_25”).
B. Valpha CDR3 Region Polypeptide Sequence:
(SEQ ID NO: 72)
CAVNANYGGATNKLIF
C. Vbeta V Region Polypeptide Sequence:
(SEQ ID NO: 73)
CAISGGEVTTYEQYF
D. Alpha Chain V(D)J Region Polynucleotide Sequence:
(SEQ ID NO: 3)
Gcccagtctgtgagccagcataaccaccacgtaattctctctgaagcagcctcactggagttgggatgcaactattcctatg
gtggaactgttaatctcttctggtatgtccagtaccctggtcaacaccttcagcttctcctcaagtacttttcaggggatcc
actggttaaaggcatcaagggctttgaggctgaatttataaagagtaaattctcctttaatctgaggaaaccctctgtgcag
tggagtgacacagctgagtacttctgtgccgtgaatgcaaattatggtggtgctacaaacaagctcatctttggaactggca
ctctgcttgctgtccagccaaat
E. Beta Chain V(D)J Region Polynucleotide Sequence:
(SEQ ID NO: 4)
Gatgctggaatcacccagagcccaagacacaaggtcacagagacaggaacaccagtgactctgagatgtcaccagactgaga
accaccgctatatgtactggtatcgacaagacccggggcatgggctgaggctgatccattactcatatggtgttaaagatac
tgacaaaggagaagtctcagatggctatagtgtctctagatcaaagacagaggatttcctcctcactctggagtccgctacc
agctcccagacatctgtgtacttctgtgccatcagtggtggggaggtaaccacctacgagcagtacttcgggccgggcacca
ggctcacggtcacagagGACCT
3. PAP-TCR-137
A. Target Peptide Sequence:
(SEQ ID NO: 47)
ILLWQPIPV (“PAP_A2_14”).
B. Valpha CDR3 Region Polypeptide Sequence:
(SEQ ID NO: 74)
CATDAPTNFGNEKLTF
C. Vbeta V Region Polypeptide Sequence:
(SEQ ID NO: 75)
CASSQRWTSGVWETQYF
D. Alpha Chain V(D)J Region Polynucleotide Sequence:
(SEQ ID NO: 5)
Agtcaacagggagaagaggatcctcaggccttgagcatccaggagggtgaaaatgccaccatgaactgcagttacaaaacta
gtataaacaatttacagtggtatagacaaaattcaggtagaggccttgtccacctaattttaatacgttcaaatgaaagaga
gaaacacagtggaagattaagagtcacgcttgacacttccaagaaaagcagttccttgttgatcacggcttcccgggcagca
gacactgcttcttacttctgtgctacggacgcccctactaactttggaaatgagaaattaacctttgggactggaacaagac
tcaccatcatacccaat
E. Beta Chain V(D)J Region Polynucleotide Sequence:
(SEQ ID NO: 6)
Gacacagctgtttcccagactccaaaatacctggtcacacagatgggaaacgacaagtccattaaatgtgaacaaaatctgg
gccatgatactatgtattggtataaacaggactctaagaaatttctgaagataatgtttagctacaataataaggagctcat
tataaatgaaacagttccaaatcgcttctcacctaaatctccagacaaagctcacttaaatcttcacatcaattccctggag
cttggtgactctgctgtgtatttctgtgccagcagccaacggtggactagcggggtgtgggagacccagtacttcgggccag
gcacgcggctcctggtgctcgagGACCT
4. PAP-TCR-149
A. Target Peptide Sequence:
(SEQ ID NO: 47)
ILLWQPIPV (“PAP_A2_14”).
B. Valpha CDR3 Region Polypeptide Sequence:
(SEQ ID NO: 76)
CAASDNNDMRF
C. Vbeta V Region Polypeptide Sequence:
(SEQ ID NO: 77)
CASSQTQGFGELFF
D. Alpha Chain V(D)J Region Polynucleotide Sequence:
(SEQ ID NO: 7)
Aaccagcaagttaagcaaaattcaccatccctgagcgtccaggaaggaagaatttctattctgaactgtgactatactaaca
gcatgtttgattatttcctatggtacaaaaaataccctgctgaaggtcctacattcctgatatctataagttccattaagga
taaaaatgaagatggaagattcactgtcttcttaaacaaaagtgccaagcacctctctctgcacattgtgccctcccagcct
ggagactctgcagtgtacttctgtgcagcaagtgataacaatgacatgcgctttggagcagggaccagactgacagtaaaac
caaat
E. Beta Chain V(D)J Region Polynucleotide Sequence:
(SEQ ID NO: 8)
Gaaacgggagttacgcagacaccaagacacctggtcatgggaatgacaaataagaagtctttgaaatgtgaacaacatctgg
gtcataacgctatgtattggtacaagcaaagtgctaagaagccactggagctcatgtttgtctacagtcttgaagaacgggt
tgaaaacaacagtgtgccaagtcgcttctcacctgaatgccccaacagctctcacttattccttcacctacacaccctgcag
ccagaagactcggccctgtatctctgcgccagcagccaaactcaggggttcggggagctgttttttggagaaggctctaggc
tgaccgtactggagGACCT
5. PAP-TCR-154
A. Target Peptide Sequence:
(SEQ ID NO: 62)
LLFFWLDRSVLA (“PAP_A2_23”).
B. Valpha CDR3 Region Polypeptide Sequence:
(SEQ ID NO: 78)
CQGAQKLVF
C. Vbeta V Region Polypeptide Sequence:
(SEQ ID NO: 79)
CASSGVGYETQYF
D. Alpha Chain V(D)J Region Polynucleotide Sequence:
(SEQ ID NO: 9)
Gctcagacagtcactcagtctcaaccagagatgtctgtgcaggaggcagagaccgtgaccctgagctgcacatatgacacca
gtgagagtgattattatttattctggtacaagcagcctcccagcaggcagatgattctcgttattcgccaagaagcttataa
gcaacagaatgcaacagagaatcgtttctctgtgaacttccagaaagcagccaaatccttcagtctcaagatctcagactca
cagctgggggatgccgcgatgtatttctgtgcttgtcagggagcccagaagctggtatttggccaaggaaccaggctgacta
tcaacccaaat
E. Beta Chain V(D)J Region Polynucleotide Sequence:
(SEQ ID NO: 10)
Gatgtgaaagtaacccagagctcgagatatctagtcaaaaggacgggagagaaagtttttctggaatgtgtccaggatatgg
accatgaaaatatgttctggtatcgacaagacccaggtctggggctacggctgatctatttctcatatgatgttaaaatgaa
agaaaaaggagatattcctgaggggtacagtgtctctagagagaagaaggagcgcttctccctgattctggagtccgccagc
accaaccagacatctatgtacctctgtgccagctcggggggggatatgagacccagtacttcgggccaggcacgcggctcct
ggtgctcgagGACCT
6. PAP-TCR-156
A. Target Peptide Sequence:
(SEQ ID NO: 48)
TLMSAMTNL (“PAP_A2_22”).
B. Valpha CDR3 Region Polypeptide Sequence:
(SEQ ID NO: 80)
CAVNNARLMF
C. Vbeta V Region Polypeptide Sequence:
(SEQ ID NO: 81)
CASSVAGSPEAFF
D. Alpha Chain V(D)J Region Polynucleotide Sequence:
(SEQ ID NO: 11)
Cagaaggaggtggagcagaattctggacccctcagtgttccagagggagccattgcctctctcaactgcacttacagtgacc
gaggttcccagtccttcttctggtacagacaatattctgggaaaagccctgagttgataatgttcatatactccaatggtga
caaagaagatggaaggtttacagcacagctcaataaagccagccagtatgtttctctgctcatcagagactcccagcccagt
gattcagccacctacctctgtgccgtgaacaatgccagactcatgtttggagatggaactcagctggtggtgaagcccaat
E. Beta Chain V(D)J Region Polynucleotide Sequence:
(SEQ ID NO: 12)
Gattctggagtcacacaaaccccaaagcacctgatcacagcaactggacagcgagtgacgctgagatgctcccctaggtctg
gagacctctctgtgtactggtaccaacagagcctggaccagggcctccagttcctcattcagtattataatggagaagagag
agcaaaaggaaacattcttgaacgattctccgcacaacagttccctgacttgcactctgaactaaacctgagctctctggag
ctgggggactcagctttgtatttctgtgccagcagcgttgcagggtcccctgaagctttctttggacaaggcaccagactca
cagttgtagagGACCT
7. PAP-TCR-168
A. Target Peptide Sequence:
(SEQ ID NO: 52)
IRSTDVDRTL (“PAP_A2_13”).
B. Valpha CDR3 Region Polypeptide Sequence:
(SEQ ID NO: 82)
CAASYPYTGRRALTF
C. Vbeta V Region Polypeptide Sequence:
(SEQ ID NO: 83)
CAASYPYTGRRALTF
D. Alpha Chain V(D)J Region Polynucleotide Sequence:
(SEQ ID NO: 13)
Ggagagaatgtggagcagcatccttcaaccctgagtgtccaggagggagacagcgctgttatcaagtgtacttattcagaca
gtgcctcaaactacttcccttggtataagcaagaacttggaaaaagacctcagcttattatagacattcgttcaaatgtggg
cgaaaagaaagaccaacgaattgctgttacattgaacaagacagccaaacatttctccctgcacatcacagagacccaacct
gaagactcggctgtctacttctgtgcagcaagttacccctacacgggcaggagagcacttacttttgggagtggaacaagac
tccaagtgcaaccaaAT
E. Beta Chain V(D)J Region Polynucleotide Sequence:
(SEQ ID NO: 14)
Gacactgaagttacccagacaccaaaacacctggtcatgggaatgacaaataagaagtctttgaaatgtgaacaacatatgg
ggcacagggctatgtattggtacaagcagaaagctaagaagccaccggagctcatgtttgtctacagctatgagaaactctc
tataaatgaaagtgtgccaagtcgcttctcacctgaatgccccaacagctctctcttaaaccttcacctacacgccctgcag
ccagaagactcagccctgtatctctgcgccagcagccaagattggggggacgagcagttcttcgggccagggacacggctca
ccgtgctagAGGACCT
8. PAP-TCR-173
A. Target Peptide Sequence:
(SEQ ID NO: 47)
ILLWQPIPV (“PAP_A2_14”).
B. Valpha CDR3 Region Polypeptide Sequence:
(SEQ ID NO: 84)
CAVEAYSGGYQKVTF
C. Vbeta V Region Polypeptide Sequence:
(SEQ ID NO: 71)
CASSMYNEQFF
D. Alpha Chain V(D)J Region Polynucleotide Sequence:
(SEQ ID NO: 15)
aaggaccaagtgtttcagccttccacagtggcatcttcagagggagctgtggtggaaatcttctgtaatcactctgtgtcca
atgcttacaacttcttctggtaccttcacttcccgggatgtgcaccaagactccttgttaaaggctcaaagccttctcagca
gggacgatacaacatGACCTatgaacggttctcttcategctgctcatcctccaggtgcgggaggcagatgctgctgtttac
tactgtgctgtggaggcatattctgggggttaccagaaagttacctttggaactggaacaaagctccaagtcatcccaaAT
E. Beta Chain V(D)J Region Polynucleotide Sequence:
(SEQ ID NO: 16)
Gatactggagtctcccagaaccccagacacaagatcacaaagaggggacagaatgtaactttcaggtgtgatccaatttctg
aacacaaccgcctttattggtaccgacagaccctggggcagggcccagagtttctgacttacttccagaatgaagctcaact
agaaaaatcaaggctgctcagtgatcggttctctgcagagaggcctaagggatctttctccaccttggagatccagcgcaca
gagcagggggactcggccatgtatctctgtgccagcagcatgtacaatgagcagttcttcgggccagggacacggctcaccg
tgctagAGGACCT
9. PAP-TCR-175
A. Target Peptide Sequence:
(SEQ ID NO: 59)
LLLARAASLSL (“PAP_A2_21”).
B. Valpha CDR3 Region Polypeptide Sequence:
(SEQ ID NO: 85)
CAFEDSGYSTLTF
C. Vbeta V Region Polypeptide Sequence:
(SEQ ID NO: 86)
CASGGLAGVDEQYF
D. Alpha Chain V(D)J Region Polynucleotide Sequence:
(SEQ ID NO: 17)
Atactgaacgtggaacaaagtcctcagtcactgcatgttcaggagggagacagcaccaatttcacctgcagcttcccttcca
gcaatttttatgccttacactggtacagatgggaaactgcaaaaagccccgaggccttgtttgtaatgactttaaatgggga
tgaaaagaagaaaggacgaataagtgccactcttaataccaaggagggttacagctatttgtacatcaaaggatcccagcct
gaagactcagccacatacctctgtgcctttgaggattcaggatacagcaccctcacctttgggaaggggactatgcttctag
tctctccagAT
E. Beta Chain V(D)J Region Polynucleotide Sequence:
(SEQ ID NO: 18)
gatgctggaatcacccagagcccaagatacaagatcacagagacaggaaggcaggtGACCTtgatgtgtcaccagacttgga
gccacagctatatgttctggtatcgacaaGACCTgggacatgggctgaggctgatctattactcagcagctgctgatattac
agataaaggagaagtccccgatggctatgttgtctccagatccaagacagagaatttccccctcactctggagtcagctacc
cgctcccagacatctgtgtatttctgcgccagcggaggactagcgggggtcgacgagcagtacttcgggccgggcaccaggc
tcacggtcacagAGGACCT
10. PAP-TCR-178
A. Target Peptide Sequence:
(SEQ ID NO: 59)
LLLARAASLSL (“PAP_A2_21”).
B. Valpha CDR3 Region Polypeptide Sequence:
(SEQ ID NO: 70)
CAASVDEKLTF
C. Vbeta V Region Polypeptide Sequence:
(SEQ ID NO: 87)
CASSSYNEQFF
D. Alpha Chain V(D)J Region Polynucleotide Sequence:
(SEQ ID NO: 19)
Gaccagcaagttaagcaaaattcaccatccctgagcgtccaggaaggaagaatttctattctgaactgtgactatactaaca
gcatgtttgattatttcctatggtacaaaaaataccctgctgaaggtcctacattcctgatatctataagttccattaagga
taaaaatgaagatggaagattcactgttttcttaaacaaaagtgccaagcacctctctctgcacattgtgccctcccagcct
ggagactctgcagtgtacttctgtgcagcaagcgtagatgagaaattaacctttgggactggaacaagactcaccatcatac
ccaAT
E. Beta Chain V(D)J Region Polynucleotide Sequence:
(SEQ ID NO: 20)
Gatactggagtctcccagaaccccagacacaagatcacaaagaggggacagaatgtaactttcaggtgtgatccaatttctg
aacacaaccgcctttattggtaccgacagaccctggggcagggcccagagtttctgacttacttccagaatgaagctcaact
agaaaaatcaaggctgctcagtgatcggttctctgcagagaggcctaagggatctttctccaccttggagatccagcgcaca
gagcagggggactcggccatgtatctctgtgccagcagctcgtacaatgagcagttcttcgggccagggacacggctcaccg
tgctagAGGACCT
11. PAP-TCR-204
A. Target Peptide Sequence:
(SEQ ID NO: 47)
ILLWQPIPV (“PAP_A2_14”).
B. Valpha CDR3 Region Polypeptide Sequence:
(SEQ ID NO: 88)
CAVGAGDYKLSF
C. Vbeta V Region Polypeptide Sequence:
(SEQ ID NO: 89)
CASSQTTGQPQHF
D. Alpha Chain V(D)J Region Polynucleotide Sequence:
(SEQ ID NO: 21)
Gcccagtcagtgacccagcctgacatccacatcactgtctctgaaggagcctcactggagttgagatgtaactattcctatg
gggcaacaccttatctcttctggtatgtccagtcccccggccaaggcctccagctgctcctgaagtacttttcaggagacac
tctggttcaaggcattaaaggctttgaggctgaatttaagaggagtcaatcttccttcaatctgaggaaaccctctgtgcat
tggagtgatgctgctgagtacttctgtgctgtgggtgccggcgactacaagctcagctttggagccggaaccacagtaactg
taagagcaaAT
E. Beta Chain V(D)J Region Polynucleotide Sequence:
(SEQ ID NO: 22)
Gaaacgggagttacgcagacaccaagacacctggtcatgggaatgacaaataagaagtctttgaaatgtgaacaacatctgg
gtcataacgctatgtattggtacaagcaaagtgctaagaagccactggagctcatgtttgtctacagtcttgaagaacgggt
tgaaaacaacagtgtgccaagtcgcttctcacctgaatgccccaacagctctcacttattccttcacctacacaccctgcag
ccagaagactcggccctgtatctctgcgccagcagccaaaccacagggcagccccagcattttggtgatgggactcgactct
ccatcctagAGGACCT
12. PAP-TCR-213
A. Target Peptide Sequence:
(SEQ ID NO: 55)
IMYSAHDTTV (“PAP_A2_25”).
B. Valpha CDR3 Region Polypeptide Sequence:
(SEQ ID NO: 90)
CAGAPETSGSRLTF
C. Vbeta V Region Polypeptide Sequence:
(SEQ ID NO: 91)
CASSFGGGSSPLHF
D. Alpha Chain V(D)J Region Polynucleotide Sequence:
(SEQ ID NO: 23)
Acccagctgctggagcagagccctcagtttctaagcatccaagagggagaaaatctcactgtgtactgcaactcctcaagtg
ttttttccagcttacaatggtacagacaggagcctggggaaggtcctgtcctcctggtgacagtagttacgggtggagaagt
gaagaagctgaagagactaacctttcagtttggtgatgcaagaaaggacagttctctccacatcactgcggcccagcctggt
gatacaggcctctacctctgtgcaggagctcccgaaaccagtggctctaggttgacctttggggaaggaacacagctcacag
tgaatcctgAT
E. Beta Chain V(D)J Region Polynucleotide Sequence:
(SEQ ID NO: 24)
Ggtgctggagtctcccagacccccagtaacaaggtcacagagaagggaaaatatgtagagctcaggtgtgatccaatttcag
gtcatactgccctttactggtaccgacaaagcctggggcagggcccagagtttctaatttacttccaaggcacgggtgcggc
agatgactcagggctgcccaacgatcggttctttgcagtcaggcctgagggatccgtctctactctgaagatccagcgcaca
gagcggggggactcagccgtgtatctctgtgccagcagcttcgggggcggaagttcacccctccactttgggaacgggacca
ggctcactgtgacagAGGACCT
13. PAP-TCR-215-1
A. Target Peptide Sequence:
(SEQ ID NO: 59)
LLLARAASLSL (“PAP_A2_21”).
B. Valpha CDR3 Region Polypeptide Sequence:
(SEQ ID NO: 92)
CAASADEKLTF
C. Vbeta V Region Polypeptide Sequence:
(SEQ ID NO: 93)
CASSQYNEQFF
D. Alpha Chain V(D)J Region Polynucleotide Sequence:
(SEQ ID NO: 25)
Gaccagcaagttaagcaaaattcaccatccctgagcgtccaggaaggaagaatttctattctgaactgtgactatactaaca
gcatgtttgattatttcctatggtacaaaaaataccctgctgaaggtcctacattcctgatatctataagttccattaagga
taaaaatgaagatggaagattcactgttttcttaaacaaaagtgccaagcacctctctctgcacattgtgccctcccagcct
ggagactctgcagtgtacttctgtgcagcaagcgcggatgagaaattaacctttgggactggaacaagactcaccatcatac
ccaAT
E. Beta Chain V(D)J Region Polynucleotide Sequence:
(SEQ ID NO: 26)
Gatactggagtctcccagaaccccagacacaagatcacaaagaggggacagaatgtaactttcaggtgtgatccaatttctg
aacacaaccgcctttattggtaccgacagaccctggggcagggcccagagtttctgacttacttccagaatgaagctcaact
agaaaaatcaaggctgctcagtgatcggttctctgcagagaggcctaagggatctttctccaccttggagatccagcgcaca
gagcagggggactcggccatgtatctctgtgccagcagccaatacaatgagcagttcttcgggccagggacacggctcaccg
tgctagAGGACCT
14. PAP-TCR-218
A. Target Peptide Sequence:
(SEQ ID NO: 59)
LLLARAASLSL (“PAP_A2_21”).
B. Valpha CDR3 Region Polypeptide Sequence:
(SEQ ID NO: 70)
CAASVDEKLTF
C. Vbeta V Region Polypeptide Sequence:
(SEQ ID NO: 94)
CASSLYNEQFF
D. Alpha Chain V(D)J Region Polynucleotide Sequence:
(SEQ ID NO: 27)
Gaccagcaagttaagcaaaattcaccatccctgagcgtccaggaaggaagaatttctattctgaactgtgactatactaaca
gcatgtttgattatttcctatggtacaaaaaataccctgctgaaggtcctacattcctgatatctataagttccattaagga
taaaaatgaagatggaagattcactgttttcttaaacaaaagtgccaagcacctctctctgcacattgtgccctcccagcct
ggagactctgcagtgtacttctgtgcagcaagcgttgatgagaaattaacctttgggactggaacaagactcaccatcatac
ccaAT
E. Beta Chain V(D)J Region Polynucleotide Sequence:
(SEQ ID NO: 28)
Gatactggagtctcccagaaccccagacacaagatcacaaagaggggacagaatgtaactttcaggtgtgatccaatttctg
aacacaaccgcctttattggtaccgacagaccctggggcagggcccagagtttctgacttacttccagaatgaagctcaact
agaaaaatcaaggctgctcagtgatcggttctctgcagagaggcctaagggatctttctccaccttggagatccagcgcaca
gagcagggggactcggccatgtatctctgtgccagcagcttatacaatgagcagttcttcgggccagggacacggctcaccg
tgctagAGGACCT
15. PAP-TCR-219
A. Target Peptide Sequence:
(SEQ ID NO: 59)
LLLARAASLSL (“PAP_A2_21”).
B. Valpha CDR3 Region Polypeptide Sequence:
(SEQ ID NO: 92)
CAASADEKLTF
C. Vbeta V Region Polypeptide Sequence:
(SEQ ID NO: 93)
CASSQYNEQFF
D. Alpha Chain V(D)J Region Polynucleotide Sequence:
(SEQ ID NO: 29)
Gaccagcaagttaagcaaaattcaccatccctgagcgtccaggaaggaagaatttctattctgaactgtgactatactaaca
gcatgtttgattatttcctatggtacaaaaaataccctgctgaaggtcctacattcctgatatctataagttccattaagga
taaaaatgaagatggaagattcactgttttcttaaacaaaagtgccaagcacctctctctgcacattgtgccctcccagcct
ggagactctgcagtgtacttctgtgcagcaagcgcggatgagaaattaacctttgggactggaacaagactcaccatcatac
ccaAT
E. Beta Chain V(D)J Region Polynucleotide Sequence:
(SEQ ID NO: 30)
Gatactggagtctcccagaaccccagacacaagatcacaaagaggggacagaatgtaactttcaggtgtgatccaatttctg
aacacaaccgcctttattggtaccgacagaccctggggcagggcccagagtttctgacttacttccagaatgaagctcaact
agaaaaatcaaggctgctcagtgatcggttctctgcagagaggcctaagggatctttctccaccttggagatccagcgcaca
gagcagggggactcggccatgtatctctgtgccagcagccaatacaatgagcagttcttcgggccagggacacggctcaccg
tgctagAGGACCT
16. PAP-TCR-220
A. Target Peptide Sequence:
(SEQ ID NO: 47)
ILLWQPIPV (“PAP_A2_14”).
B. Valpha CDR3 Region Polypeptide Sequence:
(SEQ ID NO: 95)
CAGRDNYGQNFVF
C. Vbeta V Region Polypeptide Sequence:
(SEQ ID NO: 96)
CASSQVAGGTYEQYF
D. Alpha Chain V(D)J Region Polynucleotide Sequence:
(SEQ ID NO: 31)
Ggtcaacagctgaatcagagtcctcaatctatgtttatccaggaaggagaagatgtctccatgaactgcacttcttcaagca
tatttaacacctggctatggtacaagcaggaccctggggaaggtcctgtcctcttgatagccttatataaggctggtgaatt
gacctcaaatggaagactgactgctcagtttggtataaccagaaaggacagcttcctgaatatctcagcatccatacctagt
gatgtaggcatctacttctgtgctgggcgggataactatggtcagaattttgtctttggtcccggaaccagattgtccgtgc
tgccctAT
E. Beta Chain V(D)J Region Polynucleotide Sequence:
(SEQ ID NO: 32)
Gaaacgggagttacgcagacaccaagacacctggtcatgggaatgacaaataagaagtctttgaaatgtgaacaacatctgg
ggcataacgctatgtattggtacaagcaaagtgctaagaagccactggagctcatgtttgtctacaactttaaagaacagac
tgaaaacaacagtgtgccaagtcgcttctcacctgaatgccccaacagctctcacttattccttcacctacacaccctgcag
ccagaagactcggccctgtatctctgtgccagcagccaagtggggggggaacctacgagcagtacttcgggccgggcaccag
gctcacggtcacagAGGACCT
17. PAP-TCR-223
A. Target Peptide Sequence:
(SEQ ID NO: 58)
KVYDPLYCESV (“PAP_A2_20”).
B. Valpha CDR3 Region Polypeptide Sequence:
(SEQ ID NO: 97)
CAVYGQNFVF
C. Vbeta V Region Polypeptide Sequence:
(SEQ ID NO: 98)
CASSPIGLQETQYF
D. Alpha Chain V(D)J Region Polynucleotide Sequence:
(SEQ ID NO: 33)
Cagaaggaggtggagcagaattctggacccctcagtgttccagagggagccattgcctctctcaactgcacttacagtgacc
gaggttcccagtccttcttctggtacagacaatattctgggaaaagccctgagttgataatgttcatatactccaatggtga
caaagaagatggaaggtttacagcacagctcaataaagccagccagtatgtttctctgctcatcagagactcccagcccagt
gattcagccacctacctctgtgccgtttatggtcagaattttgtctttggtcccggaaccagattgtccgtgctgccctAT
E. Beta Chain V(D)J Region Polynucleotide Sequence:
(SEQ ID NO: 34)
Gatgctggagttatccagtcaccccggcacgaggtgacagagatgggacaagaagtgactctgagatgtaaaccaatttcag
gacacgactaccttttctggtacagacagaccatgatgcggggactggagttgctcatttactttaacaacaacgttccgat
agatgattcagggatgcccgaggatcgattctcagctaagatgcctaatgcatcattctccactctgaagatccagccctca
gaacccagggactcagctgtgtacttctgtgccagcagcccaatagggctccaagagacccagtacttcgggccaggcacgc
ggctcctggtgctcgAGGACCT
18. PAP-TCR-224
A. Target Peptide Sequence:
(SEQ ID NO: 59)
LLLARAASLSL (“PAP_A2_21”).
B. Valpha CDR3 Region Polypeptide Sequence:
(SEQ ID NO: 99)
CAASEDEKLTF
C. Vbeta V Region Polypeptide Sequence:
(SEQ ID NO: 100)
CASSLMAEQYF
D. Alpha Chain V(D)J Region Polynucleotide Sequence:
(SEQ ID NO: 35)
Gaccagcaagttaagcaaaattcaccatccctgagcgtccaggaaggaagaatttctattctgaactgtgactatactaaca
gcatgtttgattatttcctatggtacaaaaaataccctgctgaaggtcctacattcctgatatctataagttccattaagga
taaaaatgaagatggaagattcactgttttcttaaacaaaagtgccaagcacctctctctgcacattgtgccctcccagcct
ggagactctgcagtgtacttctgtgcagcaagcgaggatgagaaattaacctttgggactggaacaagactcaccatcatac
ccaAT
E. Beta Chain V(D)J Region Polynucleotide Sequence:
(SEQ ID NO: 36)
Gatactggagtctcccagaaccccagacacaagatcacaaagaggggacagaatgtaactttcaggtgtgatccaatttctg
aacacaaccgcctttattggtaccgacagaccctggggcagggcccagagtttctgacttacttccagaatgaagctcaact
agaaaaatcaaggctgctcagtgatcggttctctgcagagaggcctaagggatctttctccaccttggagatccagcgcaca
gagcagggggactcggccatgtatctctgtgccagcagcttgatggcagagcagtacttcgggccgggcaccaggctcacgg
tcacagAGGACCT
19. PAP-TCR-225
A. Target Peptide Sequence:
(SEQ ID NO: 59)
LLLARAASLSL (“PAP_A2_21”).
B. Valpha CDR3 Region Polypeptide Sequence:
(SEQ ID NO: 70)
CAASVDEKLTF
C. Vbeta V Region Polypeptide Sequence:
(SEQ ID NO: 101)
CASSLQVEQFF
D. Alpha Chain V(D)J Region Polynucleotide Sequence:
(SEQ ID NO: 37)
Gaccagcaagttaagcaaaattcaccatccctgagcgtccaggaaggaagaatttctattctgaactgtgactatactaaca
gcatgtttgattatttcctatggtacaaaaaataccctgctgaaggtcctacattcctgatatctataagttccattaagga
taaaaatgaagatggaagattcactgttttcttaaacaaaagtgccaagcacctctctctgcacattgtgccctcccagcct
ggagactctgcagtgtacttctgtgcagcaagcgtcgatgagaaattaacctttgggactggaacaagactcaccatcatac
ccaAT
E. Beta Chain V(D)J Region Polynucleotide Sequence:
(SEQ ID NO: 38)
Gatactggagtctcccagaaccccagacacaagatcacaaagaggggacagaatgtaactttcaggtgtgatccaatttctg
aacacaaccgcctttattggtaccgacagaccctggggcagggcccagagtttctgacttacttccagaatgaagctcaact
agaaaaatcaaggctgctcagtgatcggttctctgcagagaggcctaagggatctttctccaccttggagatccagcgcaca
gagcagggggactcggccatgtatctctgtgccagcagcttacaggttgagcagttcttcgggccagggacacggctcaccg
tgctagAGGACCT
20. PAP-TCR-226
A. Target Peptide Sequence:
(SEQ ID NO: 59)
LLLARAASLSL (“PAP_A2_21”).
B. Valpha CDR3 Region Polypeptide Sequence:
(SEQ ID NO: 92)
CAASADEKLTF
C. Vbeta V Region Polypeptide Sequence:
(SEQ ID NO: 102)
CASSLFEEQYF
D. Alpha Chain V(D)J Region Polynucleotide Sequence:
(SEQ ID NO: 39)
Gaccagcaagttaagcaaaattcaccatccctgagcgtccaggaaggaagaatttctattctgaactgtgactatactaaca
gcatgtttgattatttcctatggtacaaaaaataccctgctgaaggtcctacattcctgatatctataagttccattaagga
taaaaatgaagatggaagattcactgttttcttaaacaaaagtgccaagcacctctctctgcacattgtgccctcccagcct
ggagactctgcagtgtacttctgtgcagcaagcgcagacgagaaattaacctttgggactggaacaagactcaccatcatac
ccaAT
E. Beta Chain V(D)J Region Polynucleotide Sequence:
(SEQ ID NO: 40)
Gatactggagtctcccagaaccccagacacaagatcacaaagaggggacagaatgtaactttcaggtgtgatccaatttctg
aacacaaccgcctttattggtaccgacagaccctggggcagggcccagagtttctgacttacttccagaatgaagctcaact
agaaaaatcaaggctgctcagtgatcggttctctgcagagaggcctaagggatctttctccaccttggagatccagcgcaca
gagcagggggactcggccatgtatctctgtgccagcagcttatttgaggagcagtacttcgggccgggcaccaggctcacgg
tcacagAGGACCT
21. PAP-TCR-228
A. Target Peptide Sequence:
(SEQ ID NO: 58)
KVYDPLYCESV (“PAP_A2_20”).
B. Valpha CDR3 Region Polypeptide Sequence:
(SEQ ID NO: 103)
CAGHLNARLMF
C. Vbeta V Region Polypeptide Sequence:
(SEQ ID NO: 104)
CSAPRDGVYTF
D. Alpha Chain V(D)J Region Polynucleotide Sequence:
(SEQ ID NO: 41)
Gctcagtcagtggctcagccggaagatcaggtcaacgttgctgaagggaatcctctgactgtgaaatgcacctattcagtct
ctggaaacccttatcttttttggtatgttcaataccccaaccgaggcctccagttccttctgaaatacatcacaggggataa
cctggttaaaggcagctatggctttgaagctgaatttaacaagagccaaacctccttccacctgaagaaaccatctgccctt
gtgagcgactccgctttgtacttctgtgctggacatctgaatgccagactcatgtttggagatggaactcagctggtggtga
agcccaAT
E. Beta Chain V(D)J Region Polynucleotide Sequence:
(SEQ ID NO: 42)
Ggtgctgtcgtctctcaacatccgagcagggttatctgtaagagtggaacctctgtgaagatcgagtgccgttccctggact
ttcaggccacaactatgttttggtatcgtcagttcccgaaacagagtctcatgctgatggcaacttccaatgagggctccaa
ggccacatacgagcaaggcgtcgagaaggacaagtttctcatcaaccatgcaagcctgaccttgtccactctgacagtgacc
agtgcccatcctgaagacagcagcttctacatctgcagtgctccccgggatggcgtatacaccttcggttcggggaccaggt
taaccgttgtagAGGACCT

### Table B: Illustrative Mutant TCR Embodiments of the Invention

The following provides illustrative examples of mutants of PAP-TCR-156. As noted in Table A above, these TCR embodiments of the invention target a PAP peptide having the sequence: TLMSAMTNL (SEQ ID NO: 48).

1.
PAP-TCR-156-4 (PAP-TCR-156-aCDR3-R7A)
A. Alpha CDR1 polypeptide sequence: DRGSQS (SEQ ID NO: 105)
B. Alpha CDR2 polypeptide sequence: IYSNGD (SEQ ID NO: 106)
C. Alpha CDR3 polypeptide sequence: CAVNNAALMF (SEQ ID NO: 107)
D. Beta CDR1 polypeptide sequence: SGDLS (SEQ ID NO: 108)
E. Beta CDR2 polypeptide sequence: YYNGEE (SEQ ID NO: 109)
F. Beta CDR3 polypeptide sequence: CASSVAGSPEAFF (SEQ ID NO: 81)
G. TCR Alpha Polynucleotide Sequence:
cagaaggaggtggagcagaattctggacccctcagtgttccagagggagccattgcctctctcaactgcacttacagtgac
cgaggttcccagtccttcttctggtacagacaatattctgggaaaagccctgagttgataatgttcatatactccaatggtgaca
aagaagatggaaggtttacagcacagctcaataaagccagccagtatgtttctctgctcatcagagactcccagcccagtga
ttcagccacctacctctgtgccgtgaacaatgccGCCctcatgtttggagatggaactcagctggtggtgaagcccaat
(SEQ ID NO: 115)
H. TCR Beta Polynucleotide Sequence:
gattctggagtcacacaaaccccaaagcacctgatcacagcaactggacagcgagtgacgctgagatgctcccctaggtc
tggagacctctctgtgtactggtaccaacagagcctggaccagggcctccagttcctcattcagtattataatggagaagag
agagcaaaaggaaacattcttgaacgattctccgcacaacagttccctgacttgcactctgaactaaacctgagctctctgga
gctgggggactcagctttgtatttctgtgccagcagcgttgcagggtcccctgaagctttctttggacaaggcaccagactca
cagttgtagagGACCT (SEQ ID NO: 116)
2.
PAP-TCR-156-29 (PAP-TCR-156-aCDR1-S4E)
A. Alpha CDR1 polypeptide sequence: DRGEQS (SEQ ID NO: 139)
B. Alpha CDR2 polypeptide sequence: IYSNGD (SEQ ID NO: 106)
C. Alpha CDR3 polypeptide sequence: CAVNNAALMF (SEQ ID NO: 107)
D. Beta CDR1 polypeptide sequence: SGDLS (SEQ ID NO: 108)
E. Beta CDR2 polypeptide sequence: YYNGEE (SEQ ID NO: 109)
F. Beta CDR3 polypeptide sequence: CASSVAGSPEAFF (SEQ ID NO: 81)
G. TCR Alpha Polynucleotide Sequence:
cagaaggaggtggagcagaattctggacccctcagtgttccagagggagccattgcctctctcaactgcacttacagtgac
cgaggtGAAcagtccttcttctggtacagacaatattctgggaaaagccctgagttgataatgttcatatactccaatggtg
acaaagaagatggaaggtttacagcacagctcaataaagccagccagtatgtttctctgctcatcagagactcccagccca
gtgattcagccacctacctctgtgccgtgaacaatgccagactcatgtttggagatggaactcagctggtggtgaagcccaa
t (SEQ ID NO: 117)
H. TCR Beta Polynucleotide Sequence:
gattctggagtcacacaaaccccaaagcacctgatcacagcaactggacagcgagtgacgctgagatgctcccctaggtc
tggagacctctctgtgtactggtaccaacagagcctggaccagggcctccagttcctcattcagtattataatggagaagag
agagcaaaaggaaacattcttgaacgattctccgcacaacagttccctgacttgcactctgaactaaacctgagctctctgga
gctgggggactcagctttgtatttctgtgccagcagcgttgcagggtcccctgaagctttctttggacaaggcaccagactca
cagttgtagagGACCT (SEQ ID NO: 118)
3.
PAP-TCR-156-30 (PAP-TCR-156-aCDR1-S6E)
A. Alpha CDR1 polypeptide sequence: DRGSQE (SEQ ID NO: 140)
B. Alpha CDR2 polypeptide sequence: IYSNGD (SEQ ID NO: 106)
C. Alpha CDR3 polypeptide sequence: CAVNNAALMF (SEQ ID NO: 107)
D. Beta CDR1 polypeptide sequence: SGDLS (SEQ ID NO: 108)
E. Beta CDR2 polypeptide sequence: YYNGEE (SEQ ID NO: 109)
F. Beta CDR3 polypeptide sequence: CASSVAGSPEAFF (SEQ ID NO: 81)
G. TCR Alpha Polynucleotide Sequence:
cagaaggaggtggagcagaattctggacccctcagtgttccagagggagccattgcctctctcaactgcacttacagtgac
cgaggttcccagGAAttcttctggtacagacaatattctgggaaaagccctgagttgataatgttcatatactccaatggtg
acaaagaagatggaaggtttacagcacagctcaataaagccagccagtatgtttctctgctcatcagagactcccagccca
gtgattcagccacctacctctgtgccgtgaacaatgccagactcatgtttggagatggaactcagctggtggtgaagcccaa
t (SEQ ID NO: 119)
H. TCR Beta Polynucleotide Sequence:
gattctggagtcacacaaaccccaaagcacctgatcacagcaactggacagcgagtgacgctgagatgctcccctaggtc
tggagacctctctgtgtactggtaccaacagagcctggaccagggcctccagttcctcattcagtattataatggagaagag
agagcaaaaggaaacattcttgaacgattctccgcacaacagttccctgacttgcactctgaactaaacctgagctctctgga
gctgggggactcagctttgtatttctgtgccagcagcgttgcagggtcccctgaagctttctttggacaaggcaccagactca
cagttgtagagGACCT (SEQ ID NO: 120)
4.
PAP-TCR-156-31 (PAP-TCR-156-aCDR1-S6H)
A. Alpha CDR1 polypeptide sequence: DRGSQH (SEQ ID NO: 141)
B. Alpha CDR2 polypeptide sequence: IYSNGD (SEQ ID NO: 106)
C. Alpha CDR3 polypeptide sequence: CAVNNAALMF (SEQ ID NO: 107)
D. Beta CDR1 polypeptide sequence: SGDLS (SEQ ID NO: 108)
E. Beta CDR2 polypeptide sequence: YYNGEE (SEQ ID NO: 109)
F. Beta CDR3 polypeptide sequence: CASSVAGSPEAFF (SEQ ID NO: 81)
G. TCR Alpha Polynucleotide Sequence:
cagaaggaggtggagcagaattctggacccctcagtgttccagagggagccattgcctctctcaactgcacttacagtgac
cgaggttcccagCACttcttctggtacagacaatattctgggaaaagccctgagttgataatgttcatatactccaatggtga
caaagaagatggaaggtttacagcacagctcaataaagccagccagtatgtttctctgctcatcagagactcccagcccagt
gattcagccacctacctctgtgccgtgaacaatgccagactcatgtttggagatggaactcagctggtggtgaagcccaat
(SEQ ID NO: 121)
H. TCR Beta Polynucleotide Sequence:
gattctggagtcacacaaaccccaaagcacctgatcacagcaactggacagcgagtgacgctgagatgctcccctaggtc
tggagacctctctgtgtactggtaccaacagagcctggaccagggcctccagttcctcattcagtattataatggagaagag
agagcaaaaggaaacattcttgaacgattctccgcacaacagttccctgacttgcactctgaactaaacctgagctctctgga
gctgggggactcagctttgtatttctgtgccagcagcgttgcagggtcccctgaagctttctttggacaaggcaccagactca
cagttgtagagGACCT (SEQ ID NO: 122)
5.
PAP-TCR-156-32 (PAP-TCR-156-aCDR1-S6N)
A. Alpha CDR1 polypeptide sequence: DRGSQN (SEQ ID NO: 142)
B. Alpha CDR2 polypeptide sequence: IYSNGD (SEQ ID NO: 106)
C. Alpha CDR3 polypeptide sequence: CAVNNAALMF (SEQ ID NO: 107)
D. Beta CDR1 polypeptide sequence: SGDLS (SEQ ID NO: 108)
E. Beta CDR2 polypeptide sequence: YYNGEE (SEQ ID NO: 109)
F. Beta CDR3 polypeptide sequence: CASSVAGSPEAFF (SEQ ID NO: 81)
G. TCR Alpha Polynucleotide Sequence:
cagaaggaggtggagcagaattctggacccctcagtgttccagagggagccattgcctctctcaactgcacttacagtgac
cgaggttcccagAATttcttctggtacagacaatattctgggaaaagccctgagttgataatgttcatatactccaatggtga
caaagaagatggaaggtttacagcacagctcaataaagccagccagtatgtttctctgctcatcagagactcccagcccagt
gattcagccacctacctctgtgccgtgaacaatgccagactcatgtttggagatggaactcagctggtggtgaagcccaat
(SEQ ID NO: 123)
H. TCR Beta Polynucleotide Sequence:
gattctggagtcacacaaaccccaaagcacctgatcacagcaactggacagcgagtgacgctgagatgctcccctaggtc
tggagacctctctgtgtactggtaccaacagagcctggaccagggcctccagttcctcattcagtattataatggagaagag
agagcaaaaggaaacattcttgaacgattctccgcacaacagttccctgacttgcactctgaactaaacctgagctctctgga
gctgggggactcagctttgtatttctgtgccagcagcgttgcagggtcccctgaagctttctttggacaaggcaccagactca
cagttgtagagGACCT (SEQ ID NO: 124)
6.
PAP-TCR-156-33 (PAP-TCR-156-aCDR2-N4H)
A. Alpha CDR1 polypeptide sequence: DRGSQS (SEQ ID NO: 105)
B. Alpha CDR2 polypeptide sequence: IYSHGD (SEQ ID NO: 143)
C. Alpha CDR3 polypeptide sequence: CAVNNAALMF (SEQ ID NO: 107)
D. Beta CDR1 polypeptide sequence: SGDLS (SEQ ID NO: 108)
E. Beta CDR2 polypeptide sequence: YYNGEE (SEQ ID NO: 109)
F. Beta CDR3 polypeptide sequence: CASSVAGSPEAFF (SEQ ID NO: 81)
G. TCR Alpha Polynucleotide Sequence:
cagaaggaggtggagcagaattctggacccctcagtgttccagagggagccattgcctctctcaactgcacttacagtgac
cgaggttcccagtccttcttctggtacagacaatattctgggaaaagccctgagttgataatgttcatatactccCACggtga
caaagaagatggaaggtttacagcacagctcaataaagccagccagtatgtttctctgctcatcagagactcccagcccagt
gattcagccacctacctctgtgccgtgaacaatgccagactcatgtttggagatggaactcagctggtggtgaagcccaat
(SEQ ID NO: 125)
H. TCR Beta Polynucleotide Sequence:
gattctggagtcacacaaaccccaaagcacctgatcacagcaactggacagcgagtgacgctgagatgctcccctaggtc
tggagacctctctgtgtactggtaccaacagagcctggaccagggcctccagttcctcattcagtattataatggagaagag
agagcaaaaggaaacattcttgaacgattctccgcacaacagttccctgacttgcactctgaactaaacctgagctctctgga
gctgggggactcagctttgtatttctgtgccagcagcgttgcagggtcccctgaagctttctttggacaaggcaccagactca
cagttgtagagGACCT (SEQ ID NO: 126)
7.
PAP-TCR-156-34 (PAP-TCR-156-aCDR2-D6N)
A. Alpha CDR1 polypeptide sequence: DRGSQS (SEQ ID NO: 105)
B. Alpha CDR2 polypeptide sequence: IYSNGN (SEQ ID NO: 106)
C. Alpha CDR3 polypeptide sequence: CAVNNAALMF (SEQ ID NO: 107)
D. Beta CDR1 polypeptide sequence: SGDLS (SEQ ID NO: 108)
E. Beta CDR2 polypeptide sequence: YYNGEE (SEQ ID NO: 109)
F. Beta CDR3 polypeptide sequence: CASSVAGSPEAFF (SEQ ID NO: 81)
G. TCR Alpha Polynucleotide Sequence:
cagaaggaggtggagcagaattctggacccctcagtgttccagagggagccattgcctctctcaactgcacttacagtgac
cgaggttcccagtccttcttctggtacagacaatattctgggaaaagccctgagttgataatgttcatatactccaatggtAA
Taaagaagatggaaggtttacagcacagctcaataaagccagccagtatgtttctctgctcatcagagactcccagcccag
tgattcagccacctacctctgtgccgtgaacaatgccagactcatgtttggagatggaactcagctggtggtgaagcccaat
(SEQ ID NO: 127)
H. TCR Beta Polynucleotide Sequence:
gattctggagtcacacaaaccccaaagcacctgatcacagcaactggacagcgagtgacgctgagatgctcccctaggtc
tggagacctctctgtgtactggtaccaacagagcctggaccagggcctccagttcctcattcagtattataatggagaagag
agagcaaaaggaaacattcttgaacgattctccgcacaacagttccctgacttgcactctgaactaaacctgagctctctgga
gctgggggactcagctttgtatttctgtgccagcagcgttgcagggtcccctgaagctttctttggacaaggcaccagactca
cagttgtagagGACCT (SEQ ID NO: 128)
8.
PAP-TCR-156-35 (PAP-TCR-156-bCDR1-S1H)
A. Alpha CDR1 polypeptide sequence: DRGSQS (SEQ ID NO: 105)
B. Alpha CDR2 polypeptide sequence: IYSNGD (SEQ ID NO: 106)
C. Alpha CDR3 polypeptide sequence: CAVNNAALMF (SEQ ID NO: 107)
D. Beta CDR1 polypeptide sequence: HGDLS (SEQ ID NO: 110)
E. Beta CDR2 polypeptide sequence: YYNGEE (SEQ ID NO: 109)
F. Beta CDR3 polypeptide sequence: CASSVAGSPEAFF (SEQ ID NO: 81)
G. TCR Alpha Polynucleotide Sequence:
cagaaggaggtggagcagaattctggacccctcagtgttccagagggagccattgcctctctcaactgcacttacagtgac
cgaggttcccagtccttcttctggtacagacaatattctgggaaaagccctgagttgataatgttcatatactccaatggtgaca
aagaagatggaaggtttacagcacagctcaataaagccagccagtatgtttctctgctcatcagagactcccagcccagtga
ttcagccacctacctctgtgccgtgaacaatgccagactcatgtttggagatggaactcagctggtggtgaagcccaat
(SEQ ID NO: 129)
H. TCR Beta Polynucleotide Sequence:
gattctggagtcacacaaaccccaaagcacctgatcacagcaactggacagcgagtgacgctgagatgctcccctaggC
ATggagacctctctgtgtactggtaccaacagagcctggaccagggcctccagttcctcattcagtattataatggagaag
agagagcaaaaggaaacattcttgaacgattctccgcacaacagttccctgacttgcactctgaactaaacctgagctctctg
gagctgggggactcagctttgtatttctgtgccagcagcgttgcagggtcccctgaagctttctttggacaaggcaccagact
cacagttgtagagGACCT (SEQ ID NO: 130)
9.
PAP-TCR-156-36 (PAP-TCR-156-bCDR1-SIN)
A. Alpha CDR1 polypeptide sequence: DRGSQS (SEQ ID NO: 105)
B. Alpha CDR2 polypeptide sequence: IYSNGD (SEQ ID NO: 106)
C. Alpha CDR3 polypeptide sequence: CAVNNAALMF (SEQ ID NO: 107)
D. Beta CDR1 polypeptide sequence: NGDLS (SEQ ID NO: 111)
E. Beta CDR2 polypeptide sequence: YYNGEE (SEQ ID NO: 109)
F. Beta CDR3 polypeptide sequence: CASSVAGSPEAFF (SEQ ID NO: 81)
G. TCR Alpha Polynucleotide Sequence:
cagaaggaggtggagcagaattctggacccctcagtgttccagagggagccattgcctctctcaactgcacttacagtgac
cgaggttcccagtccttcttctggtacagacaatattctgggaaaagccctgagttgataatgttcatatactccaatggtgaca
aagaagatggaaggtttacagcacagctcaataaagccagccagtatgtttctctgctcatcagagactcccagcccagtga
ttcagccacctacctctgtgccgtgaacaatgccagactcatgtttggagatggaactcagctggtggtgaagcccaat
(SEQ ID NO: 131)
H. TCR Beta Polynucleotide Sequence:
gattctggagtcacacaaaccccaaagcacctgatcacagcaactggacagcgagtgacgctgagatgctcccctaggA
ATggagacctctctgtgtactggtaccaacagagcctggaccagggcctccagttcctcattcagtattataatggagaag
agagagcaaaaggaaacattcttgaacgattctccgcacaacagttccctgacttgcactctgaactaaacctgagctctctg
gagctgggggactcagctttgtatttctgtgccagcagcgttgcagggtcccctgaagctttctttggacaaggcaccagact
cacagttgtagagGACCT (SEQ ID NO: 132)
10.
PAP-TCR-156-37 (PAP-TCR-156-bCDR2-Y1H)
A. Alpha CDR1 polypeptide sequence: DRGSQS (SEQ ID NO: 105)
B. Alpha CDR2 polypeptide sequence: IYSNGD (SEQ ID NO: 106)
C. Alpha CDR3 polypeptide sequence: CAVNNAALMF (SEQ ID NO: 107)
D. Beta CDR1 polypeptide sequence: SGDLS (SEQ ID NO: 108)
E. Beta CDR2 polypeptide sequence: HYNGEE (SEQ ID NO: 112)
F. Beta CDR3 polypeptide sequence: CASSVAGSPEAFF (SEQ ID NO: 81)
G. TCR Alpha Polynucleotide Sequence:
cagaaggaggtggagcagaattctggacccctcagtgttccagagggagccattgcctctctcaactgcacttacagtgac
cgaggttcccagtccttcttctggtacagacaatattctgggaaaagccctgagttgataatgttcatatactccaatggtgaca
aagaagatggaaggtttacagcacagctcaataaagccagccagtatgtttctctgctcatcagagactcccagcccagtga
ttcagccacctacctctgtgccgtgaacaatgccagactcatgtttggagatggaactcagctggtggtgaagcccaat
(SEQ ID NO: 133)
H. TCR Beta Polynucleotide Sequence:
gattctggagtcacacaaaccccaaagcacctgatcacagcaactggacagcgagtgacgctgagatgctcccctaggtc
tggagacctctctgtgtactggtaccaacagagcctggaccagggcctccagttcctcattcagCATtataatggagaag
agagagcaaaaggaaacattcttgaacgattctccgcacaacagttccctgacttgcactctgaactaaacctgagctctctg
gagctgggggactcagctttgtatttctgtgccagcagcgttgcagggtcccctgaagctttctttggacaaggcaccagact
cacagttgtagagGACCT (SEQ ID NO: 134)
11.
PAP-TCR-156-38 (PAP-TCR-156-bCDR2-N3H)
A. Alpha CDR1 polypeptide sequence: DRGSQS (SEQ ID NO: 105)
B. Alpha CDR2 polypeptide sequence: IYSNGD (SEQ ID NO: 106)
C. Alpha CDR3 polypeptide sequence: CAVNNAALMF (SEQ ID NO: 107)
D. Beta CDR1 polypeptide sequence: SGDLS (SEQ ID NO: 108)
E. Beta CDR2 polypeptide sequence: YYHGEE (SEQ ID NO: 113)
F. Beta CDR3 polypeptide sequence: CASSVAGSPEAFF (SEQ ID NO: 81)
G. TCR Alpha Polynucleotide Sequence:
cagaaggaggtggagcagaattctggacccctcagtgttccagagggagccattgcctctctcaactgcacttacagtgac
cgaggttcccagtccttcttctggtacagacaatattctgggaaaagccctgagttgataatgttcatatactccaatggtgaca
aagaagatggaaggtttacagcacagctcaataaagccagccagtatgtttctctgctcatcagagactcccagcccagtga
ttcagccacctacctctgtgccgtgaacaatgccagactcatgtttggagatggaactcagctggtggtgaagcccaat
(SEQ ID NO: 135)
H. TCR Beta Polynucleotide Sequence:
gattctggagtcacacaaaccccaaagcacctgatcacagcaactggacagegagtgacgctgagatgctcccctaggtc
tggagacctctctgtgtactggtaccaacagagcctggaccagggcctccagttcctcattcagtattatCATggagaaga
gagagcaaaaggaaacattcttgaacgattctccgcacaacagttccctgacttgcactctgaactaaacctgagctctctgg
agctgggggactcagctttgtatttctgtgccagcagcgttgcagggtcccctgaagctttctttggacaaggcaccagactc
acagttgtagagGACCT (SEQ ID NO: 136)
12.
PAP-TCR-156-39 (PAP-TCR-156-bCDR3-E10H)
A. Alpha CDR1 polypeptide sequence: DRGSQS (SEQ ID NO: 105)
B. Alpha CDR2 polypeptide sequence: IYSNGD (SEQ ID NO: 106)
C. Alpha CDR3 polypeptide sequence: CAVNNAALMF (SEQ ID NO: 107)
D. Beta CDR1 polypeptide sequence: SGDLS (SEQ ID NO: 108)
E. Beta CDR2 polypeptide sequence: YYNGEE (SEQ ID NO: 109)
F. Beta CDR3 polypeptide sequence: CASSVAGSPHAFF (SEQ ID NO: 114)
G. TCR Alpha Polynucleotide Sequence:
cagaaggaggtggagcagaattctggacccctcagtgttccagagggagccattgcctctctcaactgcacttacagtgac
cgaggttcccagtccttcttctggtacagacaatattctgggaaaagccctgagttgataatgttcatatactccaatggtgaca
aagaagatggaaggtttacagcacagctcaataaagccagccagtatgtttctctgctcatcagagactcccagcccagtga
ttcagccacctacctctgtgccgtgaacaatgccagactcatgtttggagatggaactcagctggtggtgaagcccaat
(SEQ ID NO: 137)
H. TCR Beta Polynucleotide Sequence:
gattctggagtcacacaaaccccaaagcacctgatcacagcaactggacagcgagtgacgctgagatgctcccctaggtc
tggagacctctctgtgtactggtaccaacagagcctggaccagggcctccagttcctcattcagtattataatggagaagag
agagcaaaaggaaacattcttgaacgattctccgcacaacagttccctgacttgcactctgaactaaacctgagctctctgga
gctgggggactcagctttgtatttctgtgccagcagcgttgcagggtcccctCATgctttctttggacaaggcaccagact
cacagttgtagagGACCT (SEQ ID NO: 138)

### REFERENCES

1. S. A. Rosenberg, A new era for cancer immunotherapy based on the genes that encode cancer antigens. Immunity 10, 281-287 (1999).

2. R. Srinivasan, J. D. Wolchok, Tumor antigens for cancer immunotherapy: therapeutic potential of xenogeneic DNA vaccines. J Transl Med 2, 12 (2004).
3. C. S. Hinrichs, N. P. Restifo, Reassessing target antigens for adoptive T-cell therapy. Nat Biotechnol 31, 999-1008 (2013).
4. L. A. Johnson, C. H. June, Driving gene-engineered T cell immunotherapy of cancer. Cell Res 27, 38-58 (2017).
5. R. A. Morgan et al., Cancer regression in patients after transfer of genetically engineered lymphocytes. Science 314, 126-129 (2006).
6. T. Chodon et al., Adoptive transfer of MART-1 T-cell receptor transgenic lymphocytes and dendritic cell vaccination in patients with metastatic melanoma. Clin Cancer Res 20, 2457-2465 (2014).
7. P. F. Robbins et al., Single and dual amino acid substitutions in TCR CDRs can enhance antigen-specific T cell functions. J Immunol 180, 6116-6131 (2008).
8. A. G. Chapuis et al., T cell receptor gene therapy targeting WT1 prevents acute myeloid leukemia relapse post-transplant. Nat Med 25, 1064-1072 (2019).
9. L. A. Johnson et al., Gene therapy with human and mouse T-cell receptors mediates cancer regression and targets normal tissues expressing cognate antigen. Blood 114, 535-546 (2009).
10. M. R. Parkhurst et al., T cells targeting carcinoembryonic antigen can mediate regression of metastatic colorectal cancer but induce severe transient colitis. Mol Ther 19, 620-626 (2011).
11. R. J. Rebello et al., Prostate cancer. Nat Rev Dis Primers 7, 9 (2021).
12. G. T. Consortium, The GTEx Consortium atlas of genetic regulatory effects across human tissues. Science 369, 1318-1330 (2020).
13. A. C. Jobsis, G. P. De Vries, A. E. Meijer, J. S. Ploem, The immunohistochemical detection of prostatic acid phosphatase: its possibilities and limitations in tumour histochemistry. Histochem J 13, 961-973 (1981).
14. B. Seamonds et al., Evaluation of prostate-specific antigen and prostatic acid phosphatase as prostate cancer markers. Urology 28, 472-479 (1986).
15. P. W. Kantoff et al., Sipuleucel-T immunotherapy for castration-resistant prostate cancer. N Engl J Med 363, 411-422 (2010).
16. A. S. Kibel et al., Videos of Sipuleucel-T Programmed T Cells Lysing Cells That Express Prostate Cancer Target Antigens. J Natl Cancer Inst 10.1093/jnci/djab025 (2021).
17. I. Hoof et al., NetMHCpan, a method for MHC class I binding prediction beyond humans. Immunogenetics 61, 1-13 (2009).
18. V. Jurtz et al., NetMHCpan-4.0: Improved Peptide-MHC Class I Interaction Predictions Integrating Eluted Ligand and Peptide Binding Affinity Data. J Immunol 199, 3360-3368 (2017).
19. T. J. O'Donnell et al., MHCflurry: Open-Source Class I MHC Binding Affinity Prediction. Cell Syst 7, 129-132 e124 (2018).
20 L. Fong et al., Dendritic cell-based xenoantigen vaccination for prostate cancer immunotherapy. J Immunol 167, 7150-7156 (2001).
21 J. T. Becker et al., DNA vaccine encoding prostatic acid phosphatase (PAP) elicits long-term T-cell responses in patients with recurrent prostate cancer. J Immunother 33, 639-647 (2010).
22 D. K. Wells et al., Key Parameters of Tumor Epitope Immunogenicity Revealed Through a Consortium Approach Improve Neoantigen Prediction. Cell 183, 818-834 e813 (2020).
23. W. J. Storkus, R. D. Salter, P. Cresswell, J. R. Dawson, Peptide-induced modulation of target cell sensitivity to natural killing. J Immunol 149, 1185-1190 (1992).
24. D. F. Hunt et al., Characterization of peptides bound to the class I MHC molecule HLA-A2.1 by mass spectrometry. Science 255, 1261-1263 (1992).
25 V. H. Engelhard, Structure of peptides associated with MHC class I molecules. Curr Opin Immunol 6, 13-23 (1994).
26. M. Bassani-Sternberg et al., Soluble plasma HLA peptidome as a potential source for cancer biomarkers. Proc Natl Acad Sci USA 107, 18769-18776 (2010).
27. K. A. K. Finton et al., ARTEMIS: A Novel Mass-Spec Platform for HLA-Restricted Self and Disease-Associated Peptide Discovery. Front Immunol 12, 658372 (2021).
28. K. Cao et al., Analysis of the frequencies of HLA-A, B, and C alleles and haplotypes in the five major ethnic groups of the United States reveals high levels of diversity in these loci and contrasting distribution patterns in these populations. Hum Immunol 62, 1009-1030 (2001).
29. R. Vita et al., The Immune Epitope Database (IEDB): 2018 update. Nucleic Acids Res 47, D339-D343 (2019).
30. P. A. Nesterenko et al., Droplet-based mRNA sequencing of fixed and permeabilized cells by CLInt-seq allows for antigen-specific TCR cloning. Proc Natl Acad Sci USA 118 (2021).
31. G. X. Zheng et al., Massively parallel digital transcriptional profiling of single cells. Nat Commun 8, 14049 (2017).
32. C. M. Britten et al., The use of HLA-A*0201-transfected K562 as standard antigen-presenting cells for CD8 (+) T lymphocytes in IFN-gamma ELISPOT assays. J Immunol Methods 259, 95-110 (2002).
33. A. W. Purcell, S. H. Ramarathinam, N. Ternette, Mass spectrometry-based identification of MHC-bound peptides for immunopeptidomics. Nat Protoc 14, 1687-1707 (2019).
34. T. L. Madden, R. L. Tatusov, J. Zhang, Applications of network BLAST server. Methods Enzymol 266, 131-141 (1996).
35. R. A. Henderson et al., HLA-A2.1-associated peptides from a mutant cell line: a second pathway of antigen presentation. Science 255, 1264-1266 (1992).
36. W. Chour et al., Shared Antigen-specific CD8<sup>+</sup> T cell Responses Against the SARS-COV-2 Spike Protein in HLA-A*02:01 COVID-19 Participants. medRxiv 10.1101/2020.05.04.20085779, 2020.2005.2004.20085779 (2020).
37. P. G. Maslak et al., Vaccination with synthetic analog peptides derived from WT1 oncoprotein induces T-cell responses in patients with complete remission from acute myeloid leukemia. Blood 116, 171-179 (2010).
38. T. Dao et al., Targeting the intracellular WT1 oncogene product with a therapeutic human antibody. Sci Transl Med 5, 176ra133 (2013).
39. C. G. Jakob, K. Lewinski, R. Kuciel, W. Ostrowski, L. Lebioda, Crystal structure of human prostatic acid phosphatase. Prostate 42, 211-218 (2000).
40. F. Maley, R. B. Trimble, A. L. Tarentino, T. H. Plummer, Jr., Characterization of glycoproteins and their associated oligosaccharides through the use of endoglycosidases. Anal Biochem 180, 195-204 (1989).
41. J. C. Skipper et al., An HLA-A2-restricted tyrosinase antigen on melanoma cells results from posttranslational modification and suggests a novel pathway for processing of membrane proteins. J Exp Med 183, 527-534 (1996).
42. K. K. Yu et al., Use of mutated self-cleaving 2A peptides as a molecular rheostat to direct simultaneous formation of membrane and secreted anti-HIV immunoglobulins. PLOS One 7, e50438 (2012).
43. P. Batard et al., Dextramers: new generation of fluorescent MHC class I/peptide multimers for visualization of antigen-specific CD8+ T cells. J Immunol Methods 310, 136-148 (2006).
44. S. S. Pinho, C. A. Reis, Glycosylation in cancer: mechanisms and clinical implications. Nat Rev Cancer 15, 540-555 (2015).
45. S. Mereiter, M. Balmana, D. Campos, J. Gomes, C. A. Reis, Glycosylation in the Era of Cancer-Targeted Therapy: Where Are We Heading? Cancer Cell 36, 6-16 (2019).
46. D. H. Busch, E. G. Pamer, T cell affinity maturation by selective expansion during infection. J Exp Med 189, 701-710 (1999).
47. S. Thomas et al., Human T cells expressing affinity-matured TCR display accelerated responses but fail to recognize low density of MHC-peptide antigen. Blood 118, 319-329 (2011).
48. Y. Zhao et al., High-affinity TCRs generated by phage display provide CD4+ T cells with the ability to recognize and kill tumor cell lines. J Immunol 179, 5845-5854 (2007).
49. P. D. Holler et al., In vitro evolution of a T cell receptor with high affinity for peptide/MHC. Proc Natl Acad Sci USA 97, 5387-5392 (2000).
50. L. Klein, B. Kyewski, P. M. Allen, K. A. Hogquist, Positive and negative selection of the T cell repertoire: what thymocytes see (and don't see). Nat Rev Immunol 14, 377-391 (2014).
51. C. S. Seet et al., Generation of mature T cells from human hematopoietic stem and progenitor cells in artificial thymic organoids. Nat Methods 14, 521-530 (2017).
52. S. Shukla et al., Progenitor T-cell differentiation from hematopoietic stem cells using Delta-like-4 and VCAM-1. Nat Methods 14, 531-538 (2017).
53. J. Lanoix et al., Comparison of the MHC I Immunopeptidome Repertoire of B-Cell Lymphoblasts Using Two Isolation Methods. Proteomics 18, e1700251 (2018).
54. M. Bassani-Sternberg et al., Direct identification of clinically relevant neoepitopes presented on native human melanoma tissue by mass spectrometry. Nat Commun 7, 13404 (2016).
55. J. G. Abelin et al., Mass Spectrometry Profiling of HLA-Associated Peptidomes in Mono-allelic Cells Enables More Accurate Epitope Prediction. Immunity 46, 315-326 (2017).
56. P. A. Nesterenko et al., HLA-A*02:01 restricted T cell receptors against the highly conserved SARS-COV-2 polymerase cross-react with human coronaviruses. Cell Rep 37, 110167 (2021).
57. B. Rodenko et al., Generation of peptide-MHC class I complexes through UV-mediated ligand exchange. Nat Protoc 1, 1120-1132 (2006).

1. A composition of matter comprising a polynucleotide encoding a T cell receptor (TCR) alpha chain polypeptide and/or a TCR beta chain polypeptide; wherein the polynucleotide is disposed in a vector, and when the vector is transduced into a CD8⁺ T cell, the TCR alpha chain polypeptide and/or the TCR beta chain polypeptide encoded by the polynucleotide form a T cell receptor on the CD8⁺ T cell that recognizes a polypeptide epitope of human prostatic acid phosphatase (PAP).

2. The composition of claim 1, wherein:
the T cell receptor recognizes a polypeptide epitope of human prostatic acid phosphatase in combination with human leukocyte antigen HLA-A*02:01; and/or
the T cell receptor recognizes a polypeptide epitope of human prostatic acid phosphatase selected from: ILLWQPIPV (SEQ ID NO: 47), TLMSAMTNL (SEQ ID NO: 48), IRSTDVDRTL (SEQ ID NO: 52), IMYSAHDTTV (SEQ ID NO: 55), KVYDPLYCESV (SEQ ID NO: 58), LLLARAASLSL (SEQ ID NO: 59), and LLFFWLDRSVLA (SEQ ID NO: 62).

3. The composition of claim 1, wherein the polynucleotide encodes amino acids of a TCR variable region and the vector comprises vector polynucleotides encoding a TCR constant region fused in frame with the TCR variable region.

4. The composition of claim 3, wherein the polynucleotide is disposed in a cell.

5. The composition of claim 4, wherein the cell is a human CD8⁺ T cell.

6. The composition of claim 5, wherein the CD8⁺ T cell is obtained from an individual diagnosed with a cancer that expresses a human prostatic acid phosphatase antigen; and the CD8⁺ T cell is transduced with a vector comprising a polynucleotide encoding a TCR Vα polypeptide in combination with a polynucleotide encoding a TCR Vβ polypeptide such that a heterologous TCR is expressed on a surface of the CD8⁺ T cell, wherein the heterologous TCR recognizes a human prostatic acid phosphatase peptide associated with a human leukocyte antigen expressed on the surface of cells of the cancer.

7. The composition of claim 6, wherein the cancer is a prostate cancer.

8. The composition of claim 1, wherein:
the polynucleotide encodes a segment of at least 100 amino acids having an at least 98% sequence identity to amino acids encoded by SEQ ID NO: 1-SEQ ID NO: 42.

9. The composition of claim 8, wherein the T cell receptor (TCR) alpha chain polypeptide and/or the TCR beta chain polypeptide encoded by the polynucleotide comprises an amino acid substitution mutation.

10. The composition of claim 8, wherein the polynucleotide encodes a segment of at least 10 amino acids encoded by SEQ ID NO: 115-SEQ ID NO: 138.

11. A method of inhibiting growth of a prostate cancer cell comprising:
combining the prostate cancer cell with a CD8⁺ T cell transduced with a polynucleotide encoding a T cell receptor (TCR) alpha chain polypeptide and a TCR beta chain polypeptide; wherein when transduced into and expressed in the CD8⁺ T cell, the alpha chain polypeptide and the TCR beta chain polypeptide can form a T cell receptor that recognizes a polypeptide epitope on human prostatic acid phosphatase (PAP) expressed on the prostate cancer cell, thereby inhibiting growth of the prostate cancer cell.

12. The method of claim 11, wherein:
the T cell receptor recognizes a polypeptide epitope of human prostatic acid phosphatase in combination with human leukocyte antigen HLA-A*02:01; and/or
the T cell receptor recognizes a polypeptide epitope of human prostatic acid phosphatase selected from: ILLWQPIPV (SEQ ID NO: 47), TLMSAMTNL (SEQ ID NO: 48), IRSTDVDRTL (SEQ ID NO: 52), IMYSAHDTTV (SEQ ID NO: 55), KVYDPLYCESV (SEQ ID NO: 58), LLLARAASLSL (SEQ ID NO: 59), and LLFFWLDRSVLA (SEQ ID NO: 62).

13. The method of claim 12, wherein the T cell receptor recognizes a polypeptide epitope of human prostatic acid phosphatase in combination with human leukocyte antigen HLA-A*02:01.

14. The method of claim 13, the T cell receptor recognizes a polypeptide epitope of human prostatic acid phosphatase selected from: ILLWQPIPV (SEQ ID NO: 47), TLMSAMTNL (SEQ ID NO: 48), IRSTDVDRTL (SEQ ID NO: 52), IMYSAHDTTV (SEQ ID NO: 55), KVYDPLYCESV (SEQ ID NO: 58), LLLARAASLSL (SEQ ID NO: 59), and LLFFWLDRSVLA (SEQ ID NO: 62).

15. The method of claim 14, the T cell receptor recognizes a polypeptide epitope of human prostatic acid phosphatase comprising TLMSAMTNL (SEQ ID NO: 48).

16. The method of claim 11, wherein CD8⁺ T cells are combined in vivo so as to treat an individual suffering from prostate cancer.

17. The method of claim 12, wherein the polynucleotide the polynucleotide encodes a segment of at least 100 amino acids having an at least 98% sequence identity to amino acids encoded by SEQ ID NO: 1-SEQ ID NO: 42.

18. The method of claim 17, wherein the T cells express a T cell receptor that comprises a segment of at least 10 amino acids encoded by SEQ ID NO: 115-SEQ ID NO: 138.

19. A method of assessing a patient immune response to a prostate cancer or prostate cancer vaccination, the method comprising observing the induction or activation of T cells obtained from a patient having a prostate cancer or prostate cancer vaccination, wherein:
the induction or activation of T cells is observed in response to the T cell's exposure to a polypeptide epitope present on human prostatic acid phosphatase (PAP); and
an observed induction or activation of T cells provides evidence of patient immune response to prostate cancer or prostate cancer vaccination.

20. The method of claim 19, wherein the T cells express a T cell receptor that recognizes a polypeptide epitope of human prostatic acid phosphatase selected from:
(SEQ ID NO: 47)
ILLWQPIPV,
(SEQ ID NO: 48)
TLMSAMTNL,
(SEQ ID NO: 52)
IRSTDVDRTL,
(SEQ ID NO: 55)
IMYSAHDTTV,
(SEQ ID NO: 58)
KVYDPLYCESV,
(SEQ ID NO: 59)
LLLARAASLSL,
and
(SEQ ID NO: 62)
LLFFWLDRSVLA.


SEQUENCE LISTING (148 sequences)

SEQ ID NO: 1 - DNA, 333 bp, synthetic construct
  source 1..333: mol_type = other DNA
gaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgttttcttaaacaaaagtgccaagcacctctctctgcacattgtgccctcccagcctggagactctgcagtgtacttctgtgcagcaagcgtagatgagaaattaacctttgggactggaacaagactcaccatcatacccaat

SEQ ID NO: 2 - DNA, 341 bp, synthetic construct
  source 1..341: mol_type = other DNA
gatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcatgtacaatgagcagttcttcgggccagggacacggctcaccgtgctagaggacct

SEQ ID NO: 3 - DNA, 351 bp, synthetic construct
  source 1..351: mol_type = other DNA
gcccagtctgtgagccagcataaccaccacgtaattctctctgaagcagcctcactggagttgggatgcaactattcctatggtggaactgttaatctcttctggtatgtccagtaccctggtcaacaccttcagcttctcctcaagtacttttcaggggatccactggttaaaggcatcaagggctttgaggctgaatttataaagagtaaattctcctttaatctgaggaaaccctctgtgcagtggagtgacacagctgagtacttctgtgccgtgaatgcaaattatggtggtgctacaaacaagctcatctttggaactggcactctgcttgctgtccagccaaat

SEQ ID NO: 4 - DNA, 350 bp, synthetic construct
  source 1..350: mol_type = other DNA
gatgctggaatcacccagagcccaagacacaaggtcacagagacaggaacaccagtgactctgagatgtcaccagactgagaaccaccgctatatgtactggtatcgacaagacccggggcatgggctgaggctgatccattactcatatggtgttaaagatactgacaaaggagaagtctcagatggctatagtgtctctagatcaaagacagaggatttcctcctcactctggagtccgctaccagctcccagacatctgtgtacttctgtgccatcagtggtggggaggtaaccacctacgagcagtacttcgggccgggcaccaggctcacggtcacagaggacct

SEQ ID NO: 5 - DNA, 345 bp, synthetic construct
  source 1..345: mol_type = other DNA
agtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacggacgcccctactaactttggaaatgagaaattaacctttgggactggaacaagactcaccatcatacccaat

SEQ ID NO: 6 - DNA, 356 bp, synthetic construct
  source 1..356: mol_type = other DNA
gacacagctgtttcccagactccaaaatacctggtcacacagatgggaaacgacaagtccattaaatgtgaacaaaatctgggccatgatactatgtattggtataaacaggactctaagaaatttctgaagataatgtttagctacaataataaggagctcattataaatgaaacagttccaaatcgcttctcacctaaatctccagacaaagctcacttaaatcttcacatcaattccctggagcttggtgactctgctgtgtatttctgtgccagcagccaacggtggactagcggggtgtgggagacccagtacttcgggccaggcacgcggctcctggtgctcgaggacct

SEQ ID NO: 7 - DNA, 333 bp, synthetic construct
  source 1..333: mol_type = other DNA
aaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaagtgataacaatgacatgcgctttggagcagggaccagactgacagtaaaaccaaat

SEQ ID NO: 8 - DNA, 347 bp, synthetic construct
  source 1..347: mol_type = other DNA
gaaacgggagttacgcagacaccaagacacctggtcatgggaatgacaaataagaagtctttgaaatgtgaacaacatctgggtcataacgctatgtattggtacaagcaaagtgctaagaagccactggagctcatgtttgtctacagtcttgaagaacgggttgaaaacaacagtgtgccaagtcgcttctcacctgaatgccccaacagctctcacttattccttcacctacacaccctgcagccagaagactcggccctgtatctctgcgccagcagccaaactcaggggttcggggagctgttttttggagaaggctctaggctgaccgtactggaggacct

SEQ ID NO: 9 - DNA, 339 bp, synthetic construct
  source 1..339: mol_type = other DNA
gctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcttgtcagggagcccagaagctggtatttggccaaggaaccaggctgactatcaacccaaat

SEQ ID NO: 10 - DNA, 344 bp, synthetic construct
  source 1..344: mol_type = other DNA
gatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctctgtgccagctcgggggtgggatatgagacccagtacttcgggccaggcacgcggctcctggtgctcgaggacct

SEQ ID NO: 11 - DNA, 327 bp, synthetic construct
  source 1..327: mol_type = other DNA
cagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgtgaacaatgccagactcatgtttggagatggaactcagctggtggtgaagcccaat

SEQ ID NO: 12 - DNA, 344 bp, synthetic construct
  source 1..344: mol_type = other DNA
gattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcgttgcagggtcccctgaagctttctttggacaaggcaccagactcacagttgtagaggacct

SEQ ID NO: 13 - DNA, 345 bp, synthetic construct
  source 1..345: mol_type = other DNA
ggagagaatgtggagcagcatccttcaaccctgagtgtccaggagggagacagcgctgttatcaagtgtacttattcagacagtgcctcaaactacttcccttggtataagcaagaacttggaaaaagacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcaagttacccctacacgggcaggagagcacttacttttgggagtggaacaagactccaagtgcaaccaaat

SEQ ID NO: 14 - DNA, 344 bp, synthetic construct
  source 1..344: mol_type = other DNA
gacactgaagttacccagacaccaaaacacctggtcatgggaatgacaaataagaagtctttgaaatgtgaacaacatatggggcacagggctatgtattggtacaagcagaaagctaagaagccaccggagctcatgtttgtctacagctatgagaaactctctataaatgaaagtgtgccaagtcgcttctcacctgaatgccccaacagctctctcttaaaccttcacctacacgccctgcagccagaagactcagccctgtatctctgcgccagcagccaagattggggggacgagcagttcttcgggccagggacacggctcaccgtgctagaggacct

SEQ ID NO: 15 - DNA, 327 bp, synthetic construct
  source 1..327: mol_type = other DNA
aaggaccaagtgtttcagccttccacagtggcatcttcagagggagctgtggtggaaatcttctgtaatcactctgtgtccaatgcttacaacttcttctggtaccttcacttcccgggatgtgcaccaagactccttgttaaaggctcaaagccttctcagcagggacgatacaacatgacctatgaacggttctcttcatcgctgctcatcctccaggtgcgggaggcagatgctgctgtttactactgtgctgtggaggcatattctgggggttaccagaaagttacctttggaactggaacaaagctccaagtcatcccaaat

SEQ ID NO: 16 - DNA, 341 bp, synthetic construct; identical to SEQ ID NO: 2
  source 1..341: mol_type = other DNA

SEQ ID NO: 17 - DNA, 339 bp, synthetic construct
  source 1..339: mol_type = other DNA
atactgaacgtggaacaaagtcctcagtcactgcatgttcaggagggagacagcaccaatttcacctgcagcttcccttccagcaatttttatgccttacactggtacagatgggaaactgcaaaaagccccgaggccttgtttgtaatgactttaaatggggatgaaaagaagaaaggacgaataagtgccactcttaataccaaggagggttacagctatttgtacatcaaaggatcccagcctgaagactcagccacatacctctgtgcctttgaggattcaggatacagcaccctcacctttgggaaggggactatgcttctagtctctccagat

SEQ ID NO: 18 - DNA, 347 bp, synthetic construct
  source 1..347: mol_type = other DNA
gatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtccccgatggctatgttgtctccagatccaagacagagaatttccccctcactctggagtcagctacccgctcccagacatctgtgtatttctgcgccagcggaggactagcgggggtcgacgagcagtacttcgggccgggcaccaggctcacggtcacagaggacct

SEQ ID NO: 19 - DNA, 333 bp, synthetic construct; identical to SEQ ID NO: 1
  source 1..333: mol_type = other DNA

SEQ ID NO: 20 - DNA, 341 bp, synthetic construct
  source 1..341: mol_type = other DNA
gatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagctcgtacaatgagcagttcttcgggccagggacacggctcaccgtgctagaggacct

SEQ ID NO: 21 - DNA, 339 bp, synthetic construct
  source 1..339: mol_type = other DNA
gcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgccggcgactacaagctcagctttggagccggaaccacagtaactgtaagagcaaat

SEQ ID NO: 22 - DNA, 344 bp, synthetic construct
  source 1..344: mol_type = other DNA
gaaacgggagttacgcagacaccaagacacctggtcatgggaatgacaaataagaagtctttgaaatgtgaacaacatctgggtcataacgctatgtattggtacaagcaaagtgctaagaagccactggagctcatgtttgtctacagtcttgaagaacgggttgaaaacaacagtgtgccaagtcgcttctcacctgaatgccccaacagctctcacttattccttcacctacacaccctgcagccagaagactcggccctgtatctctgcgccagcagccaaaccacagggcagccccagcattttggtgatgggactcgactctccatcctagaggacct

SEQ ID NO: 23 - DNA, 339 bp, synthetic construct
  source 1..339: mol_type = other DNA
acccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcggcccagcctggtgatacaggcctctacctctgtgcaggagctcccgaaaccagtggctctaggttgacctttggggaaggaacacagctcacagtgaatcctgat

SEQ ID NO: 24 - DNA, 350 bp, synthetic construct
  source 1..350: mol_type = other DNA
ggtgctggagtctcccagacccccagtaacaaggtcacagagaagggaaaatatgtagagctcaggtgtgatccaatttcaggtcatactgccctttactggtaccgacaaagcctggggcagggcccagagtttctaatttacttccaaggcacgggtgcggcagatgactcagggctgcccaacgatcggttctttgcagtcaggcctgagggatccgtctctactctgaagatccagcgcacagagcggggggactcagccgtgtatctctgtgccagcagcttcgggggcggaagttcacccctccactttgggaacgggaccaggctcactgtgacagaggacct

SEQ ID NO: 25 - DNA, 333 bp, synthetic construct
  source 1..333: mol_type = other DNA
gaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgttttcttaaacaaaagtgccaagcacctctctctgcacattgtgccctcccagcctggagactctgcagtgtacttctgtgcagcaagcgcggatgagaaattaacctttgggactggaacaagactcaccatcatacccaat

SEQ ID NO: 26 - DNA, 341 bp, synthetic construct
  source 1..341: mol_type = other DNA
gatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagccaatacaatgagcagttcttcgggccagggacacggctcaccgtgctagaggacct

SEQ ID NO: 27 - DNA, 333 bp, synthetic construct
  source 1..333: mol_type = other DNA
gaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgttttcttaaacaaaagtgccaagcacctctctctgcacattgtgccctcccagcctggagactctgcagtgtacttctgtgcagcaagcgttgatgagaaattaacctttgggactggaacaagactcaccatcatacccaat

SEQ ID NO: 28 - DNA, 341 bp, synthetic construct
  source 1..341: mol_type = other DNA
gatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcttatacaatgagcagttcttcgggccagggacacggctcaccgtgctagaggacct

SEQ ID NO: 29 - DNA, 333 bp, synthetic construct; identical to SEQ ID NO: 25
  source 1..333: mol_type = other DNA

SEQ ID NO: 30 - DNA, 341 bp, synthetic construct; identical to SEQ ID NO: 26
  source 1..341: mol_type = other DNA

SEQ ID NO: 31 - DNA, 336 bp, synthetic construct
  source 1..336: mol_type = other DNA
ggtcaacagctgaatcagagtcctcaatctatgtttatccaggaaggagaagatgtctccatgaactgcacttcttcaagcatatttaacacctggctatggtacaagcaggaccctggggaaggtcctgtcctcttgatagccttatataaggctggtgaattgacctcaaatggaagactgactgctcagtttggtataaccagaaaggacagcttcctgaatatctcagcatccatacctagtgatgtaggcatctacttctgtgctgggcgggataactatggtcagaattttgtctttggtcccggaaccagattgtccgtgctgccctat

SEQ ID NO: 32 - DNA, 350 bp, synthetic construct
  source 1..350: mol_type = other DNA
gaaacgggagttacgcagacaccaagacacctggtcatgggaatgacaaataagaagtctttgaaatgtgaacaacatctggggcataacgctatgtattggtacaagcaaagtgctaagaagccactggagctcatgtttgtctacaactttaaagaacagactgaaaacaacagtgtgccaagtcgcttctcacctgaatgccccaacagctctcacttattccttcacctacacaccctgcagccagaagactcggccctgtatctctgtgccagcagccaagtggcggggggaacctacgagcagtacttcgggccgggcaccaggctcacggtcacagaggacct

SEQ ID NO: 33 - DNA, 327 bp, synthetic construct
  source 1..327: mol_type = other DNA
cagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgtttatggtcagaattttgtctttggtcccggaaccagattgtccgtgctgccctat

SEQ ID NO: 34 - DNA, 350 bp, synthetic construct
  source 1..350: mol_type = other DNA
gatgctggagttatccagtcaccccggcacgaggtgacagagatgggacaagaagtgactctgagatgtaaaccaatttcaggacacgactaccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagcccaatagggctccaagagacccagtacttcgggccaggcacgcggctcctggtgctcgaggacct

SEQ ID NO: 35 - DNA, 333 bp, synthetic construct
  source 1..333: mol_type = other DNA
gaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgttttcttaaacaaaagtgccaagcacctctctctgcacattgtgccctcccagcctggagactctgcagtgtacttctgtgcagcaagcgaggatgagaaattaacctttgggactggaacaagactcaccatcatacccaat

SEQ ID NO: 36 - DNA, 341 bp, synthetic construct
  source 1..341: mol_type = other DNA
gatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcttgatggcagagcagtacttcgggccgggcaccaggctcacggtcacagaggacct

SEQ ID NO: 37 - DNA, 333 bp, synthetic construct
  source 1..333: mol_type = other DNA
gaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgttttcttaaacaaaagtgccaagcacctctctctgcacattgtgccctcccagcctggagactctgcagtgtacttctgtgcagcaagcgtcgatgagaaattaacctttgggactggaacaagactcaccatcatacccaat

SEQ ID NO: 38 - DNA, 341 bp, synthetic construct
  source 1..341: mol_type = other DNA
gatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcttacaggttgagcagttcttcgggccagggacacggctcaccgtgctagaggacct

SEQ ID NO: 39 - DNA, 333 bp, synthetic construct
  source 1..333: mol_type = other DNA
gaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgttttcttaaacaaaagtgccaagcacctctctctgcacattgtgccctcccagcctggagactctgcagtgtacttctgtgcagcaagcgcagacgagaaattaacctttgggactggaacaagactcaccatcatacccaat

SEQ ID NO: 40 - DNA, 341 bp, synthetic construct
  source 1..341: mol_type = other DNA
gatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcttatttgaggagcagtacttcgggccgggcaccaggctcacggtcacagaggacct

SEQ ID NO: 41 - DNA, 336 bp, synthetic construct
  source 1..336: mol_type = other DNA
gctcagtcagtggctcagccggaagatcaggtcaacgttgctgaagggaatcctctgactgtgaaatgcacctattcagtctctggaaacccttatcttttttggtatgttcaataccccaaccgaggcctccagttccttctgaaatacatcacaggggataacctggttaaaggcagctatggctttgaagctgaatttaacaagagccaaacctccttccacctgaagaaaccatctgcccttgtgagcgactccgctttgtacttctgtgctggacatctgaatgccagactcatgtttggagatggaactcagctggtggtgaagcccaat

SEQ ID NO: 42 - DNA, 347 bp, synthetic construct
  source 1..347: mol_type = other DNA
ggtgctgtcgtctctcaacatccgagcagggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctccccgggatggcgtatacaccttcggttcggggaccaggttaaccgttgtagaggacct

SEQ ID NO: 43 - protein, 8 aa, Homo sapiens
KELKFVTL

SEQ ID NO: 44 - protein, 9 aa, Homo sapiens
FQKRLHPYK

SEQ ID NO: 45 - protein, 9 aa, Homo sapiens
LSGLHGQDL

SEQ ID NO: 46 - protein, 9 aa, Homo sapiens
FQKRLHPYK

SEQ ID NO: 47 - protein, 9 aa, Homo sapiens
ILLWQPIPV

SEQ ID NO: 48 - protein, 9 aa, Homo sapiens
TLMSAMTNL

SEQ ID NO: 49 - protein, 9 aa, Homo sapiens
VLAKELKFV

SEQ ID NO: 50 - protein, 10 aa, Homo sapiens
MEQHYELGEY

SEQ ID NO: 51 - protein, 10 aa, Homo sapiens
GEYFVEMYYR

SEQ ID NO: 52 - protein, 10 aa, Homo sapiens
IRSTDVDRTL

SEQ ID NO: 53 - protein, 10 aa, Homo sapiens
IWSKVYDPLY

SEQ ID NO: 54 - protein, 10 aa, Homo sapiens
SVHNFTLPSW

SEQ ID NO: 55 - protein, 10 aa, Homo sapiens
IMYSAHDTTV

SEQ ID NO: 56 - protein, 11 aa, Homo sapiens
DFIATLGKLSG

SEQ ID NO: 57 - protein, 11 aa, Homo sapiens
DVYNGLLPPYA

SEQ ID NO: 58 - protein, 11 aa, Homo sapiens
KVYDPLYCESV

SEQ ID NO: 59 - protein, 11 aa, Homo sapiens
LLLARAASLSL

SEQ ID NO: 60 - protein, 12 aa, Homo sapiens
SPIDTFPTDPIK

SEQ ID NO: 61 - protein, 12 aa, Homo sapiens
WQPIPVHTVPLS

SEQ ID NO: 62 - protein, 12 aa, Homo sapiens
LLFFWLDRSVLA

SEQ ID NO: 63 - protein, 13 aa, Homo sapiens
YSAHDTTVSGLQM

SEQ ID NO: 64 - protein, 14 aa, Homo sapiens
YSAHDTTVSGLQMA

SEQ ID NO: 65 - protein, 14 aa, Homo sapiens
LSELSLLSLYGIHK

SEQ ID NO: 66 - protein, 14 aa, Homo sapiens
IATLGKLSGLHGQD

SEQ ID NO: 67 - protein, 14 aa, Homo sapiens
KELKFVTLVFRHGD

SEQ ID NO: 68 - protein, 15 aa, Homo sapiens
IATLGKLSGLHGQDL

SEQ ID NO: 69 - protein, 10 aa, synthetic construct
SVHDFTLPSW

SEQ ID NO: 70 - protein, 11 aa, synthetic construct
CAASVDEKLTF

SEQ ID NO: 71 - protein, 11 aa, synthetic construct
CASSMYNEQFF

SEQ ID NO: 72 - protein, 16 aa, synthetic construct
CAVNANYGGATNKLIF

SEQ ID NO: 73 - protein, 15 aa, synthetic construct
CAISGGEVTTYEQYF

SEQ ID NO: 74 - protein, 16 aa, synthetic construct
CATDAPTNFGNEKLTF

SEQ ID NO: 75 - protein, 17 aa, synthetic construct
CASSQRWTSGVWETQYF

SEQ ID NO: 76 - protein, 11 aa, synthetic construct
CAASDNNDMRF

SEQ ID NO: 77 - protein, 14 aa, synthetic construct
CASSQTQGFGELFF

SEQ ID NO: 78 - protein, 9 aa, synthetic construct
CQGAQKLVF

SEQ ID NO: 79 - protein, 13 aa, synthetic construct
CASSGVGYETQYF

SEQ ID NO: 80 - protein, 10 aa, synthetic construct
CAVNNARLMF

SEQ ID NO: 81 - protein, 13 aa, synthetic construct
CASSVAGSPEAFF

SEQ ID NO: 82 - protein, 15 aa, synthetic construct
CAASYPYTGRRALTF

SEQ ID NO: 83 - protein, 15 aa, synthetic construct
CAASYPYTGRRALTF

SEQ ID NO: 84 - protein, 15 aa, synthetic construct
CAVEAYSGGYQKVTF

SEQ ID NO: 85 - protein, 13 aa, synthetic construct
CAFEDSGYSTLTF

SEQ ID NO: 86 - protein, 14 aa, synthetic construct
CASGGLAGVDEQYF

SEQ ID NO: 87 - protein, 11 aa, synthetic construct
CASSSYNEQFF

SEQ ID NO: 88 - protein, 12 aa, synthetic construct
CAVGAGDYKLSF

SEQ ID NO: 89 - protein, 13 aa, synthetic construct
CASSQTTGQPQHF

SEQ ID NO: 90 - protein, 14 aa, synthetic construct
CAGAPETSGSRLTF

SEQ ID NO: 91 - protein, 14 aa, synthetic construct
CASSFGGGSSPLHF

SEQ ID NO: 92 - protein, 11 aa, synthetic construct
CAASADEKLTF

SEQ ID NO: 93 - protein, 11 aa, synthetic construct
CASSQYNEQFF

SEQ ID NO: 94 - protein, 11 aa, synthetic construct
CASSLYNEQFF

SEQ ID NO: 95 - protein, 13 aa, synthetic construct
CAGRDNYGQNFVF

SEQ ID NO: 96 - protein, 15 aa, synthetic construct
CASSQVAGGTYEQYF

SEQ ID NO: 97 - protein, 10 aa, synthetic construct
CAVYGQNFVF

SEQ ID NO: 98 - protein, 14 aa, synthetic construct
CASSPIGLQETQYF

SEQ ID NO: 99 - protein, 11 aa, synthetic construct
CAASEDEKLTF

SEQ ID NO: 100 - protein, 11 aa, synthetic construct
CASSLMAEQYF

SEQ ID NO: 101 - protein, 11 aa, synthetic construct
CASSLQVEQFF

SEQ ID NO: 102 - protein, 11 aa, synthetic construct
CASSLFEEQYF

SEQ ID NO: 103 - protein, 11 aa, synthetic construct
CAGHLNARLMF

SEQ ID NO: 104 - protein, 11 aa, synthetic construct
CSAPRDGVYTF

SEQ ID NO: 105 - protein, 6 aa, synthetic construct
DRGSQS

SEQ ID NO: 106 - protein, 6 aa, synthetic construct
IYSNGD

SEQ ID NO: 107 - protein, 10 aa, synthetic construct
CAVNNAALMF

SEQ ID NO: 108 - protein, 5 aa, synthetic construct
SGDLS

SEQ ID NO: 109 - protein, 6 aa, synthetic construct
YYNGEE

SEQ ID NO: 110 - protein, 5 aa, synthetic construct
HGDLS

SEQ ID NO: 111 - protein, 5 aa, synthetic construct
NGDLS

SEQ ID NO: 112 - protein, 6 aa, synthetic construct
HYNGEE

SEQ ID NO: 113 - protein, 6 aa, synthetic construct
YYHGEE

SEQ ID NO: 114 - protein, 13 aa, synthetic construct
CASSVAGSPHAFF

SEQ ID NO: 115 - DNA, 327 bp, synthetic construct
  source 1..327: mol_type = other DNA
cagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgtgaacaatgccgccctcatgtttggagatggaactcagctggtggtgaagcccaat

SEQ ID NO: 116 - DNA, 344 bp, synthetic construct; identical to SEQ ID NO: 12
  source 1..344: mol_type = other DNA

SEQ ID NO: 117 - DNA, 327 bp, synthetic construct
  source 1..327: mol_type = other DNA
cagaaggaggtggagcagaattctggacccctcagtgttccagagggagccattgcctctctcaactgcacttacagtgaccgaggtgaacagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgtgaacaatgccagactcatgtttggagatggaactcagctggtggtgaagcccaat

SEQ ID NO: 118 - DNA, 344 bp, synthetic construct; identical to SEQ ID NO: 12
  source 1..344: mol_type = other DNA

SEQ ID NO: 119 - DNA, 327 bp, synthetic construct
  source 1..327: mol_type = other DNA
cagaaggaggtggagcagaattctggacccctcagtgttccagagggagccattgcctctctcaactgcacttacagtgaccgaggttcccaggaattcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgtgaacaatgccagactcatgtttggagatggaactcagctggtggtgaagcccaat

SEQ ID NO: 120 - DNA, 344 bp, synthetic construct; identical to SEQ ID NO: 12
  source 1..344: mol_type = other DNA

SEQ ID NO: 121 - DNA, 327 bp, synthetic construct
  source 1..327: mol_type = other DNA
cagaaggaggtggagcagaattctggacccctcagtgttccagagggagccattgcctctctcaactgcacttacagtgaccgaggttcccagcacttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgtgaacaatgccagactcatgtttggagatggaactcagctggtggtgaagcccaat

SEQ ID NO: 122 - DNA, 344 bp, synthetic construct; identical to SEQ ID NO: 12
  source 1..344: mol_type = other DNA

SEQ ID NO: 123 - DNA, 327 bp, synthetic construct
  source 1..327: mol_type = other DNA
cagaaggaggtggagcagaattctggacccctcagtgttccagagggagccattgcctctctcaactgcacttacagtgaccgaggttcccagaatttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgtgaacaatgccagactcatgtttggagatggaactcagctggtggtgaagcccaat

SEQ ID NO: 124 - DNA, 344 bp, synthetic construct; identical to SEQ ID NO: 12
  source 1..344: mol_type = other DNA

SEQ ID NO: 125 - DNA, 327 bp, synthetic construct
  source 1..327: mol_type = other DNA
cagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactcccacggtgacaaagaagatggaaggtttacagcacagctcaataaagccagccagtatgtttctctgctcatcagagactcccagcccagtgattcagccacctacctctgtgccgtgaacaatgccagactcatgtttggagatggaactcagctggtggtgaagcccaat

SEQ ID NO: 126 - DNA, 344 bp, synthetic construct; identical to SEQ ID NO: 12
  source 1..344: mol_type = other DNA

SEQ ID NO: 127 - DNA, 327 bp, synthetic construct
  source 1..327: mol_type = other DNA
cagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtaataaagaagatggaaggtttacagcacagctcaataaagccagccagtatgtttctctgctcatcagagactcccagcccagtgattcagccacctacctctgtgccgtgaacaatgccagactcatgtttggagatggaactcagctggtggtgaagcccaat

SEQ ID NO: 128 - DNA, 344 bp, synthetic construct; identical to SEQ ID NO: 12
  source 1..344: mol_type = other DNA

SEQ ID NO: 129 - DNA, 327 bp, synthetic construct; identical to SEQ ID NO: 11
  source 1..327: mol_type = other DNA

SEQ ID NO: 130 - DNA, 344 bp, synthetic construct
  source 1..344: mol_type = other DNA
gattctggagtcacacaaaccccaaagcacctgatcacagcaactggacagcgagtgacgctgagatgctcccctaggcatggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcgttgcagggtcccctgaagctttctttggacaaggcaccagactcacagttgtagaggacct

SEQ ID NO: 131 - DNA, 327 bp, synthetic construct; identical to SEQ ID NO: 11
  source 1..327: mol_type = other DNA

SEQ ID NO: 132 - DNA, 344 bp, synthetic construct
  source 1..344: mol_type = other DNA
gattctggagtcacacaaaccccaaagcacctgatcacagcaactggacagcgagtgacgctgagatgctcccctaggaatggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcgttgcagggtcccctgaagctttctttggacaaggcaccagactcacagttgtagaggacct

SEQ ID NO: 133 - DNA, 327 bp, synthetic construct; identical to SEQ ID NO: 11
  source 1..327: mol_type = other DNA

SEQ ID NO: 134 - DNA, 344 bp, synthetic construct
  source 1..344: mol_type = other DNA
gattctggagtcacacaaaccccaaagcacctgatcacagcaactggacagcgagtgacgctgagatgctcccctaggtctggagacctctctgtgtactggtaccaacagagcctggaccagggcctccagttcctcattcagcattataatggagaagagagagcaaaaggaaacattcttgaacgattctccgcacaacagttccctgacttgcactctgaactaaacctgagctctctggagctgggggactcagctttgtatttctgtgccagcagcgttgcagggtcccctgaagctttctttggacaaggcaccagactcacagttgtagaggacct

SEQ ID NO: 135 - DNA, 327 bp, synthetic construct; identical to SEQ ID NO: 11
  source 1..327: mol_type = other DNA

SEQ ID NO: 136 - DNA, 344 bp, synthetic construct
  source 1..344: mol_type = other DNA
gattctggagtcacacaaaccccaaagcacctgatcacagcaactggacagcgagtgacgctgagatgctcccctaggtctggagacctctctgtgtactggtaccaacagagcctggaccagggcctccagttcctcattcagtattatcatggagaagagagagcaaaaggaaacattcttgaacgattctccgcacaacagttccctgacttgcactctgaactaaacctgagctctctggagctgggggactcagctttgtatttctgtgccagcagcgttgcagggtcccctgaagctttctttggacaaggcaccagactcacagttgtagaggacct

SEQ ID NO: 137 - DNA, 327 bp, synthetic construct; identical to SEQ ID NO: 11
  source 1..327: mol_type = other DNA

SEQ ID NO: 138 - DNA, 344 bp, synthetic construct
  source 1..344: mol_type = other DNA
gattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcgttgcagggtcccctcatgctttctttggacaaggcaccagactcacagttgtagaggacct

SEQ ID NO: 139 - protein, 6 aa, synthetic construct
DRGEQS

SEQ ID NO: 140 - protein, 6 aa, synthetic construct
DRGSQE

SEQ ID NO: 141 - protein, 6 aa, synthetic construct
DRGSQH

SEQ ID NO: 142 - protein, 6 aa, synthetic construct
DRGSQN

SEQ ID NO: 143 - protein, 6 aa, synthetic construct
IYSHGD

SEQ ID NO: 144 - protein, 6 aa, synthetic construct
HHHHHH

SEQ ID NO: 145 - protein, 9 aa, Homo sapiens
RMFPNAPYL

SEQ ID NO: 146 - protein, 10 aa, unidentified
EAAGIGILTV

SEQ ID NO: 147 - protein, 11 aa, Homo sapiens
  SITE 1: note = amidated residue
  SITE 11: note = Carboxylated residue
LLLARAASLSL

SEQ ID NO: 148 - protein, 10 aa, Homo sapiens
  SITE 1: note = amidated residue
  SITE 10: note = Carboxylated residue
SVHNFTLPSW